(19) United States
(12) Patent Application Publication
(10) Pub. No.: US 2009/0163696 A1
(43) Pub. Date: Jun. 25, 2009

(54) METHOD FOR PREPARING LYSOBACTIN DERIVATIVES

(76) Inventors: Franz VON NUSSBAUM, Duesseldorf (DE); Sonja Anlauf, Wuppertal (DE); Johannes Koebberling, Grevenbroich (DE); Joachim Telser, Wuppertal (DE); Dieter Haebich, Wuppertal (DE)

Correspondence Address: MORRISON & FOERSTER LLP, 12531 HIGH BLUFF DRIVE, SUITE 100, SAN DIEGO, CA, 92130-2040, US

(21) Appl. No.: 12/249,880

(22) Filed: Oct. 10, 2008

Related U.S. Application Data
(63) Continuation of application No. PCT/EP2007/003313, filed on Apr. 13, 2007.

(30) Foreign Application Priority Data
Apr. 13, 2006 (DE) DE 102006018250.2

(51) Int. Cl. C07K 11/00; C07K 1/06

(52) U.S. Cl. 530/329; 530/335

(57) ABSTRACT

Method for preparing cyclic depsipeptides having the following formula (I)

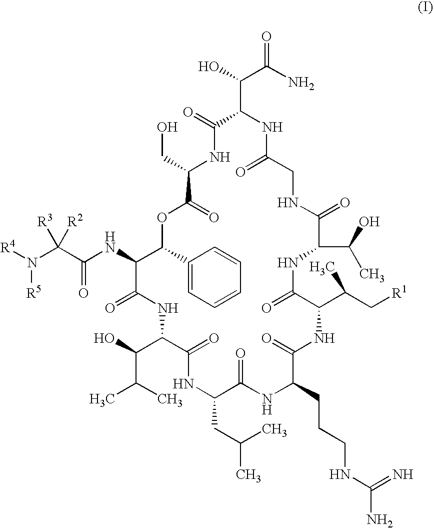

by intramolecular cyclization.

### CROSS-REFERENCE TO RELATED APPLICATIONS

This application is a continuation of pending international application PCT/EP2007/003313, filed Apr. 13, 2007, designating US, which claims priority from German patent application DE 10 2006 018 250.2, filed Apr. 13, 2006. The contents of these documents are incorporated herein by reference in their entirety.

### BACKGROUND OF THE INVENTION

The present invention relates to a method for preparing cyclic depsipeptides of the following formula (I)

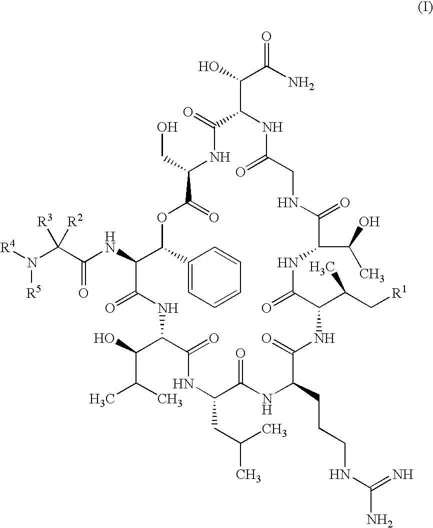

in which R¹ is H or CH₃,

in which R² is hydrogen, C₃-C₆-cycloalkyl, C₅-C₆-cycloalkenyl, C₃-C₆ cycloalkylmethyl, 5- to 7-membered heterocyclylmethyl, methyl, ethyl, n-propyl, isopropyl, 1-methylprop-1-yl, 2-methylprop-1-yl, 2,2-dimethylprop-1-yl, 1,1-dimethylprop-1-yl, 1-ethylprop-1-yl, 1-ethyl-1-methylprop-1-yl, n-butyl, 2-methylbut-1-yl, 3-methylbut-1-yl, 1-ethylbut-1-yl, tert-butyl, 4-methylpent-1-yl, n-hexyl, alkenyl or aryl,

whereby R² may be substituted with 0, 1, 2 or 3 substituents selected independently of one another from the group consisting of halogen, hydroxy, amino, cyano, trimethylsilyl, alkyl, alkoxy, benzyloxy, C₃-C₆-cycloalkyl, aryl, 5- to 10-membered heteroaryl, alkylamino, arylamino, alkylcarbonylamino, arylcarbonylamino, alkylcarbonyl, alkoxycarbonyl, arylcarbonyl and benzyloxycarbonylamino,

wherein aryl and heteroaryl in turn may be substituted with 0, 1, 2 or 3 substituents selected independently of one another from the group consisting of halogen, hydroxy, amino, cyano, nitro, alkyl, alkoxy and phenyl,

in which R³ is hydrogen or C₁-C₄-alkyl,

or

in which R² and R³ together with the carbon atom to which they are bonded form a C₃-C₆-cycloalkyl ring or a 5- to 7-membered heterocyclyl ring, whereby the cycloalkyl ring and the heterocyclyl ring may be substituted with 0, 1, 2 or 3 substituents selected independently of one another from the group consisting of trifluoromethyl, alkyl, alkoxy and alkylcarbonyl,

in which R⁴ is alkyl, C₃-C₆-cycloalkyl, 5- to 7-membered heterocyclyl, aryl, 5- or 6-membered heteroaryl, alkylcarbonyl, alkoxycarbonyl, C₃-C₆-cycloalkylcarbonyl, 5- to 7-membered heterocyclylcarbonyl, arylcarbonyl, 5- or 6-membered heteroarylcarbonyl or alkylaminocarbonyl,

whereby alkyl, cycloalkyl, heterocyclyl, aryl, heteroaryl, alkoxycarbonyl, cycloalkylcarbonyl, heterocyclylcarbonyl, arylcarbonyl, heteroarylcarbonyl and alkylaminocarbonyl may be substituted with 0, 1, 2 or 3 substituents selected independently of one another from the group consisting of halogen, hydroxy, amino, alkylamino and phenyl,

and

whereby alkylcarbonyl is substituted with an amino or alkylamino substituent,

and

whereby alkylcarbonyl may be substituted with a further 0, 1 or 2 substituents selected independently of one another from the group consisting of halogen, hydroxy, trimethylsilyl, alkoxy, alkylthio, benzyloxy, C₃-C₆-cycloalkyl, phenyl, naphthyl, 5- to 10-membered heteroaryl, alkylcarbonylamino, alkoxycarbonylamino, arylcarbonylamino, arylcarbonyloxy, benzyloxycarbonyl and benzyloxycarbonylamino,

whereby phenyl and heteroaryl in turn may be substituted with 0, 1, 2 or 3 substituents selected independently of one another from the group consisting of halogen, hydroxy, nitro, alkyl, alkoxy and phenyl,

or two substituents on the same carbon atom in the alkylcarbonyl together with the carbon atom to which they are bonded form a C₃-C₆-cycloalkyl ring or a 5- to 7-membered heterocyclyl ring,

whereby the cycloalkyl ring and the heterocyclyl ring may be substituted with 0, 1, 2 or 3 substituents selected independently of one another from the group consisting of trifluoromethyl, alkyl and alkoxy,

or

whereby the cycloalkyl ring may be benzo-fused,

in which R⁵ is hydrogen, C₁-C₄-alkyl, cyclopropyl or cyclopropylmethyl,

or

in which R⁴ and R⁵ together with the nitrogen atom to which they are bonded form a 5- to 7-membered heterocyclyl ring, whereby the heterocyclyl ring may be substituted with 0, 1, 2, or 3 substituents selected independently of one another from the group consisting of halogen, hydroxy, amino, cyano, alkyl, alkoxy and alkylamino,

as well as compounds useful in this method.

The cyclic depsipeptides depicted above include inter alia the two natural products depicted below, which are referred to as lysobactin and katanosin A. These substances are inhibitors of the cell wall biosynthesis and thus have antibacterial activity.

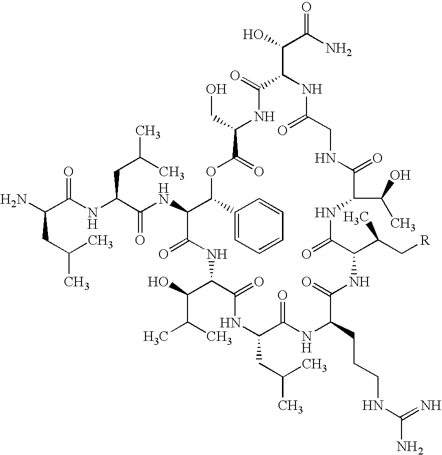

lysobactin, R═CH₃

katanosin A, R═H

The bacterial cell wall is synthesized by a number of enzymes (cell wall biosynthesis) and is essential for the survival and reproduction of microorganisms. The structure of this macromolecule, as well as the proteins and biosynthesis intermediates (“precursor”) involved in the synthesis thereof, are highly conserved within the bacteria. Owing to its essential nature and uniformity, cell wall biosynthesis is an ideal point of attack for novel antibiotics.

Vancomycin and penicillin are inhibitors of the bacterial cell wall biosynthesis and represent successful examples of the antibiotic potency of this principle of action. They have been employed for several decades clinically for the treatment of bacterial infections, especially with gram-positive pathogens. Due to the growing occurrence of resistant microbes, for example methicillin-resistant staphylococci, penicillin-resistant pneumococci and vancomycin-resistant enterococci, and recently also for the first time vancomycin-resistant staphylococci, these substances are increasingly losing their therapeutic efficacy.

Lysobactin has to date been obtained by fermentation using for example Lysobacter sp. SC 14067. It is further known from WO 2004/099239 A1 to remove the two leucine units which form the linear segment and replace them with other groups. A way of preparing lysobactin, katanosin A or derivatives thereof having variations in the linear segment by complete synthesis is not known to date.

### SUMMARY OF THE INVENTION

It is thus an object of the invention to describe a method for preparing cyclic depsipeptides of the abovementioned formula (I).

This object is achieved by a method for preparing cyclic depsipeptides of formula (I) by intramolecular cyclization of a compound of the following formula (II)

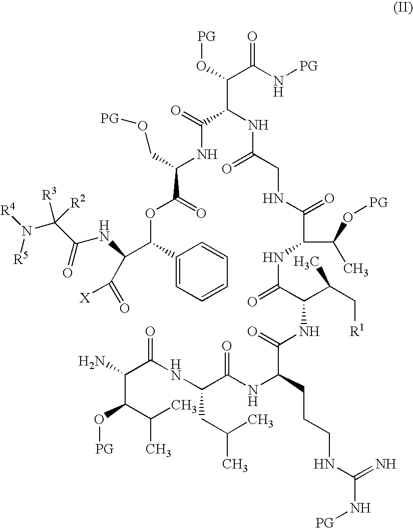

in which R¹ to R⁵ are as defined above,

in which X is OH, an active ester, a pseudohalogen (e.g. an azide) or a halogen, and

in which PG is H or a suitable protecting group,

and subsequent deprotection of the cyclic intermediate to form the cyclic depsipeptide of formula (I).

This method is characterized in that the β-hydroxy α-amino acid (here β-phenylserine=β-hydroxyphenylalanine)esterified on O³ is chemically activated in the cyclization step and then behaves as acyl donor. The cyclization takes place by an amide linkage (lactam formation) and not by an esterification reaction (lactone formation).

The method is further characterized in that the cyclic segment of the depsipeptide lasso structure is prepared by a cyclization at the bridgehead amino acid (here β-hydroxyphenylalanine).

For the purpose of the present invention, substituents have the following meaning unless specified otherwise:

Alkyl per se and “alk” and “alkyl” in alkoxy, alkylamino, alkylcarbonyl, alkoxycarbonyl, alkylaminocarbonyl and alkylcarbonylamino represents a linear or branched alkyl radical generally having 1 to 6, preferably 1 to 4, particularly preferably 1 to 3 carbon atoms, by way of example and preferably methyl, ethyl, n-propyl, isopropyl, tert-butyl, n-pentyl and n-hexyl.

Alkoxy by way of example and preferably represents methoxy, ethoxy, n-propoxy, isopropoxy, tert-butoxy, n-pentoxy and n-hexoxy.

Alkenyl represents a straight-chain or branched alkenyl radical having 2 to 6 carbon atoms. Preference is given to a straight-chain or branched alkenyl radical having 2 to 4, particularly preferably having 2 to 3 carbon atoms. Examples which may be preferably mentioned are: vinyl, allyl, n-prop-1-en-1-yl, n-but-2-en-1-yl, 2-methylprop-1-en-1-yl and 2-methylprop-2-en-1-yl.

Alkylamino represents an alkylamino radical having one or two alkyl substituents (chosen independently of one another), by way of example and preferably methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Arylamino represents an arylamino radical having one aryl substituent and optionally a further substituent such as, for example, aryl or alkyl, by way of example and preferably phenylamino, naphthylamino, phenylmethylamino or diphenylamino.

Alkylcarbonyl represents an alkylcarbonyl radical having one alkyl substituent, by way of example and preferably methylcarbonyl, ethylcarbonyl, n-propylcarbonyl, isopropylcarbonyl, tert-butylcarbonyl, n-pentylcarbonyl and n-hexylcarbonyl.

Alkoxycarbonyl represents an alkoxycarbonyl radical having one alkoxy substituent, by way of example and preferably methoxycarbonyl, ethoxycarbonyl, n-propoxycarbonyl, isopropoxycarbonyl, tert-butoxycarbonyl, n-pentoxycarbonyl and n-hexoxycarbonyl.

Cycloalkylcarbonyl represents a cycloalkylcarbonyl radical having one cycloalkyl substituent, by way of example and preferably cyclopropylcarbonyl, cyclobutylcarbonyl, cyclopentylcarbonyl and cyclohexylcarbonyl.

Heterocyclylcarbonyl represents a heterocyclylcarbonyl radical having one heterocyclyl substituent, by way of example and preferably tetrahydrofuran-2-ylcarbonyl, pyrrolidin-2-ylcarbonyl, pyrrolidin-3-ylcarbonyl, pyrrolinylcarbonyl, piperidinylcarbonyl, morpholinylcarbonyl and perhydroazepinylcarbonyl.

Arylcarbonyl represents an arylcarbonyl radical having one aryl substituent, by way of example and preferably phenylcarbonyl, naphthylcarbonyl and phenanthrenylcarbonyl.

Heteroarylcarbonyl represents a heteroarylcarbonyl radical having one heteroaryl substituent, by way of example and preferably thienylcarbonyl, furylcarbonyl, pyrrolylcarbonyl, thiazolylcarbonyl, oxazolylcarbonyl, imidazolylcarbonyl, pyridylcarbonyl, pyrimidylcarbonyl, pyridazinylcarbonyl, indolylcarbonyl, indazolylcarbonyl, benzofuranylcarbonyl, benzothiophenylcarbonyl, quinolinylcarbonyl and isoquinolinylcarbonyl.

Alkylcarbonylamino represents an alkylcarbonylamino radical having one alkyl substituent, by way of example and preferably methylcarbonylamino, ethylcarbonylamino, n-propylcarbonylamino, isopropylcarbonylamino, tert-butylcarbonylamino, n-pentylcarbonylamino and n-hexylcarbonylamino.

Arylcarbonylamino represents an arylcarbonylamino radical having one aryl substituent, by way of example and preferably phenylcarbonylamino, naphthylcarbonylamino and phenanthrenylcarbonylamino.

Alkylaminocarbonyl represents an alkylaminocarbonyl radical having one or two alkyl substituents (chosen independently of one another), by way of example and preferably methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-tert-butyl-N-methylaminocarbonyl, N-ethyl-N-n-pentylaminocarbonyl and N-n-hexyl-N-methylaminocarbonyl.

Cycloalkyl represents a cycloalkyl group generally having 3 to 6 carbon atoms, by way of example and preferably cyclopropyl, cyclobutyl, cyclopentyl and cyclohexyl.

Cycloalkenyl represents a cycloalkenyl group generally having 5 to 6 carbon atoms and one or two double bonds, by way of example and preferably cyclopent-1-en-1-yl, cyclopent-2-en-1-yl, cyclopent-3-en-1-yl, cyclohex-1-en-1-yl, cyclohex-2-en-1-yl and cyclohex-3-en-1-yl.

Aryl represents a mono- to tricyclic aromatic, carbocyclic radical generally having 6 to 14 carbon atoms; by way of example and preferably phenyl, naphthyl and phenanthrenyl.

Heterocyclyl represents a mono- or polycyclic, preferably mono- or bicyclic, heterocyclic radical generally having 5 to 7 ring atoms and up to 3, preferably up to 2 heteroatoms and/or hetero groups from the series N, O, S, SO, SO₂. The heterocyclyl radicals may be saturated or partly unsaturated. Preference is given to 5- to 7-membered monocyclic saturated heterocyclyl radicals having up to two heteroatoms from the series O, N and S, such as by way of example and preferably tetrahydrofuran-2-yl, pyrrolidin-2-yl, pyrrolidin-3-yl, pyrrolinyl, piperidinyl, morpholinyl and perhydroazepinyl.

Heteroaryl represents an aromatic, mono- or bicyclic radical generally having 5 to 10, preferably 5 to 6 ring atoms and up to 5, preferably up to 4 heteroatoms from the series S, O and N, by way of example and preferably thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl and isoquinolinyl.

Carbonyl-bonded amino acid represents an amino acid which is bonded via the carbonyl group of the amino acid function. Preference is given in this connection to α-amino acids in the L- or in the D-configuration, in particular naturally occurring α-amino acids such as, for example, glycine, L-alanine, L-valine, L-leucine, L-isoleucine, L-proline, L-phenylalanine, L-tryptophan or naturally occurring α-amino acids in the unnatural D-configuration such as, for example, D-alanine, D-valine, D-leucine, D-isoleucine, D-proline, D-phenylalanine, D-tryptophan or unnatural amino acids having a side group bonded to the α-carbon atom of the amino acid, such as, for example, C₃-C₆-cycloalkylmethyl, C₃-C₆-cycloalkyl, ethyl, n-propyl, 2,2-dimethylpropyl, tert-butyl, 3-methylbutyl, n-hexyl or allyl, or the side chain forms a ring with the α-carbon atom of the amino acid such as, for example, cyclopropyl (amino acid: 1-amino-1-cyclopropanecarboxylic acid), cyclobutyl, cyclopentyl or cyclohexyl, or β-amino acids (for nomenclature, cf.: D. Seebach, M. Overhand, F. N. M. Kühnle, B. Martinoni, L. Oberer, U. Hommel, H. Widmer, Helv. Chim. Acta 1996, 79, 913-941), such as, for example, β-alanine, β-phenylalanine, β-Aib (α-methylalanine) or derivatives of 2,3-diaminopropionic acid (e.g. 2,3-diamino-3-phenylpropionic acid).

Halogen represents fluorine, chlorine, bromine and iodine, preferably fluorine and chlorine.

The term active ester includes all active esters known to the man of the art. Examples of active esters preferred in the invention include cyanomethyl esters, p-nitrophenyl esters, o-nitrophenyl esters, 2,4-dinitrophenyl esters, 2,4,5-trichlorophenyl esters, pentachlorophenyl esters, pentafluorophenyl esters (Pfp), N-hydroxyphthalimide esters, N-hydroxysuccinimide esters (O-Su), 1-hydroxypiperidine esters, 5-chloro-8-hydroxyquinoline esters.

The intramolecular cyclization involves the formation of an amide bond which can in principle be achieved by any process known to a man of the art.

The cyclization thereby takes place by the nucleophilic attack depicted below.

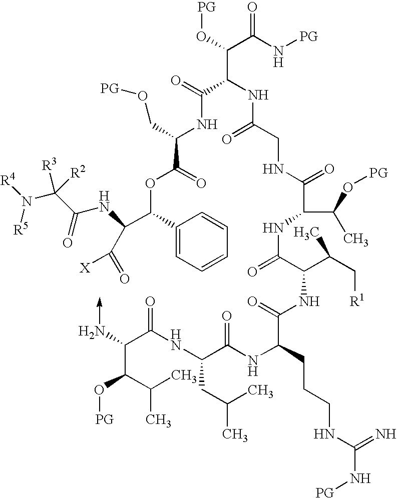

If X represents an active ester, a pseudohalogen or a halogen, the reaction generally takes place in inert solvents, where appropriate in the presence of a base, preferably in a temperature range from −30° C. to 50° under atmospheric pressure.

Examples of inert solvents are tetrahydrofuran, methylene chloride, pyridine, dioxane, chloroform, diethyl ether, tert-butyl methyl ether, ethyl acetate or dimethylformamide, with preference for methylene chloride or dimethyl formamide.

Examples of bases are triethylamine, triisopropylethylamine or N-methylmorpholine, with preference for triisopropylethylamine.

If X represents OH, the reaction generally takes place in inert solvents in the presence of a dehydrating reagent, where appropriate in the presence of a base, preferably in a temperature range from −30° C. to 50° C. under atmospheric pressure.

Examples of inert solvents are halohydrocarbons such as dichloromethane or trichloromethane, hydrocarbons such as benzene, nitromethane, dioxane, dimethylformamide or acetonitrile. It is also possible to employ a mixture of solvents. Particularly preferred solvents are dichloromethane and dimethylformamide.

Examples of suitable dehydrating reagents are carbodiimides such as, for example, N,N′-diethyl-, N,N-dipropyl-, N,N-diisopropyl-, N,N-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole or 1,2-oxazolium compounds such as 2-ethyl-5-phenyl-1,2-oxazolium 3-sulfate or 2-tert-butyl-5-methyl-isooxazolium perchlorate or acylamino compounds such as 2-ethoxy-1-ethoxycarbonyl-1,2-dihydroquinoline or propanephosphonic anhydride or isobutyl chloroformate or bis(2-oxo-3-oxazolidinyl)phosphoryl chloride or benzotriazyloxytri(dimethylamino)phosphonium hexafluorophosphate or O-(benzotriazol-1-yl)-N,N,N′,N′-tetramethyluronium hexafluorophosphate (HBTU), 2-(2-oxo-1-(2-H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU) or 1-hydroxybenzotriazole (HOBt) or benzotriazol-1-yloxytris(dimethylamino)phosphonium hexafluorophosphate (BOP) or benzotriazol-1-yloxytris(pyrrolidino)phosphonium hexafluorophosphate (PyBOP) or N-hydroxysuccinimide or mixtures thereof with bases.

Examples of bases are alkali metal carbonates such as, for example, sodium or potassium carbonate or bicarbonate or organic bases such as trialkylamines, e.g. triethylamine, N-methylmorpholine, 4-methylmorpholine, N-methylpiperidine, 4-dimethylaminopyridine or diisopropylethylamine.

The reaction is preferably carried out with HATU in the presence of 4-methylmorpholine.

The compounds of formula (II) carry protecting groups where appropriate, so that in these cases the intramolecular cyclization of the compound of formula (II) is followed by a removal of the protecting groups by methods known to the man of the art.

The term “suitable protecting group” as used herein includes all protecting groups which are known to a man of the art and can be used to mask a specific function and can thereafter be removed again without initiating further alterations in the molecule to be deprotected.

For example, primary or secondary hydroxy groups can be protected as cleavable ethers, in particular as methoxymethyl, benzyloxymethyl, p-methoxybenzyloxymethyl, benzyl, tert-butyl, tetrahydropyranyl, allyl, p-chlorophenyl, p-nitrophenyl or triphenylmethyl ethers. Silyl ethers represent a further possibility for protecting hydroxy groups, for example trimethylsilyl (TMS), tert-butyldimethylsilyl (TBDMS), triisopropylsilyl (TIPS), tert-butyldiphenylsilyl (TBDPS) or triphenylsilyl ethers. Hydroxy groups can further also be protected by ester groups, for example by acetyl, benzoyl, propionyl, chloroacetyl, trichloroacetyl, trifluoroacetyl, or crotyl esters. Besides these, carbonates, such as, for example, methyl carbonate, allyl carbonate, benzyl carbonate are also suitable for protecting alcohols. It is further possible to use esters of sulfuric acid or sulfonic acids such as, for example, sulfate, allylsulfonate, p-toluenesulfonate (tosylate) or methylsulfonate as protecting groups for alcohols.

Preferred protecting groups for hydroxy groups are tert-butyl ethers or silyl ethers, especially tert-butyldimethylsilyl ethers.

Protecting groups suitable for the guanidino group are in principle the same as for hydroxy groups, with preference in this case for the (2,2,5,7,8-pentamethyl-3,4-dihydro-2H-chromen-6-yl)sulfonyl group (PMC group).

Carboxy groups can be protected in the form of their alkyl, silyl, arylalkyl or arylesters, for example as methyl, ethyl, tert-butyl, trimethylsilyl, tert-butyldimethylsilyl, benzyl, picolyl, trichloroethyl or trimethylsilyl esters. Carboxy groups can also be protected in the form of various amides, anilides or hydrazides, for example as N,N-dimethylamide, pyrrolidinylamide, piperidinylamide, o-nitroanilide, N-7-nitroindolylamide or N-phenylhydrazide. Besides these, they can also be protected as orthoesters, for example as trimethyl orthoesters. Carboxylic acids are preferably protected in the form of their esters, especially as methyl or trimethylsilylethyl esters.

Groups particularly suitable for protecting amino groups are those which afford cleavable carbamates, for example methoxycarbonyl, tert-butoxycarbonyl (Boc), benzyloxycarbonyl (CBz or Z), allyloxycarbonyl (alloc), 9-fluoroenylmethoxycarbonyl (Fmoc), 2-trimethylsilylethylcarbonyl, 1-adamantylcarbonyl, m-nitrophenyl groups. Amino groups can further also be protected in the form of easily cleavable amides or imides, for example as formamide, chloroacetamide, trichloroacetamide, trifluoroacetamide, benzoylamide, o-nitrophenylacetamide, phthalimide, tetrachlorophthalimide or nitrophthalimide. A further possibility for protecting amino groups is to form cleavable amines with particular alkyl groups such as, for example, the tert-butyl group, the methyl group, the triphenylmethyl group, the ferrocenylmethyl group or the allyl group or with aryl groups such as, for example, the 2,4-dinitrophenyl group.

Carbamates are preferably used to protect the amino group, and among these in particular tert-butoxycarbonyl (Boc), benzyloxycarbonyl (CBz or Z) or 9-fluoroenylmethoxycarbonyl groups (Fmoc).

The protecting groups listed here serve merely as example and do not represent an exhaustive listing of all the possibilities. A more extensive treatment of this area is to be found inter alia in T. W. Greene, P. G. M. Wuts Protective Groups in Organic Synthesis, 3^{rd} Edition, John Wiley & Sons Inc. 1999.

The groups represented by PG as used herein may in one molecule be the same or different suitable protecting groups or combinations of identical or different protecting groups with H or exclusively H.

The compound of formula (II) is preferably a compound of the following formula (IIa)

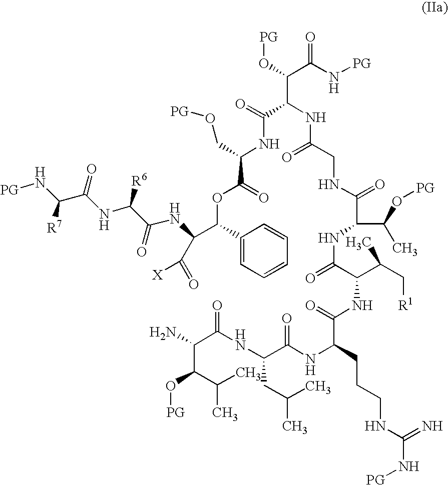

in which X and R¹ are as defined above,

in which R⁶ is isopropylmethyl, tert-butylmethyl, 2,2-dimethylbut-1-yl, 2-ethyl-2-methylbut-1-yl, 2,2-diethylbut-1-yl, 2,2-dimethylpent-1-yl, 3-pyridylmethyl, 4-trifluoromethyl-3-pyridylmethyl, benzyl or trimethylsilylmethyl,

in which R⁷ is isopropylmethyl, tert-butylmethyl, 2,2-dimethylbut-1-yl, 2-ethyl-2-methylbut-1-yl, 2,2-diethylbut-1-yl, 2,2-dimethylpent-1-yl, trimethylsilylmethyl or benzyl, and

in which PG is H or a suitable protecting group.

R⁶ is preferably isopropylmethyl, tert-butylmethyl or 3-pyridylmethyl and in particular R⁶ is isopropylmethyl.

R⁷ is preferably isopropylmethyl, tert-butylmethyl or trimethylsilylmethyl and in particular R⁷ is isopropylmethyl.

X is preferably OH.

R¹ is preferably CH₃.

In a further embodiment of the invention, the compound (II) is prepared by coupling a compound of the following formula (III) with a compound of the following formula (IV)

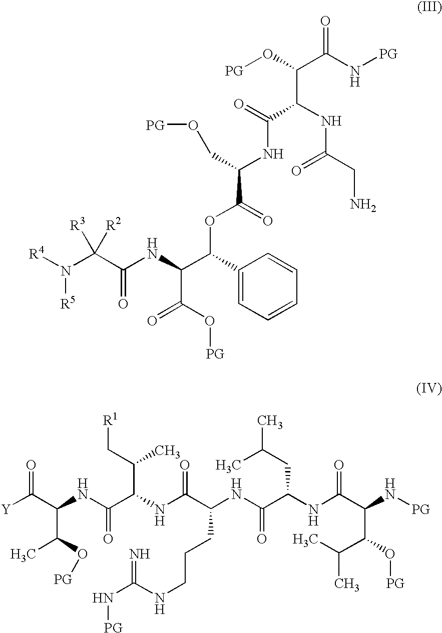

in which R¹ to R⁵ are as defined above,

in which Y is OH, an active ester, a pseudohalogen or a halogen, and

in which PG is H or a suitable protecting group,

and, where appropriate, partial or complete deprotection of the intermediate, and where appropriate conversion of the carboxy group of the 3-hydroxyphenylalanine into a group of formula —C(═O)X in which X is as defined above.

The compound of formula (III) is in particular a compound of the following formula (IIa)

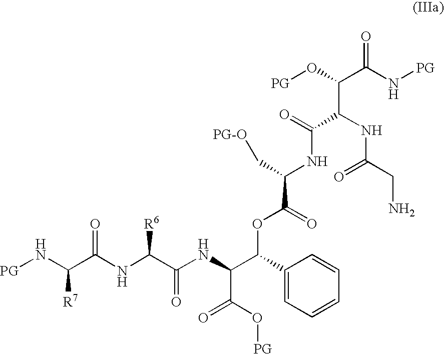

in which R⁶ and R⁷ are as defined above, and

in which PG is H or a suitable protecting group.

The coupling can take place under the same or different conditions as described above for the intramolecular cyclization. The coupling thereby takes place by the nucleophilic attack depicted below.

The conversion of the carboxy group of the 3-hydroxyphenylalanine into a group of formula —C(═O)X can take place by methods known to a man of the art.

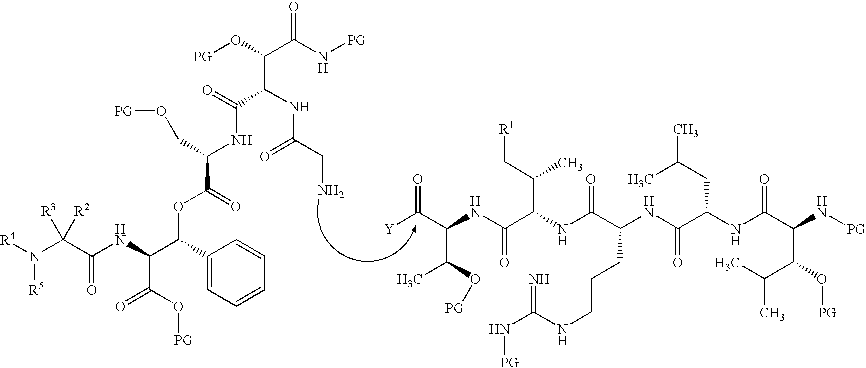

The protecting groups present in the intermediate may correspond entirely, partly or not at all to those of the desired product. These can be removed, replaced or attached where appropriate by methods known to a man of the art.

In a further embodiment of the invention, a compound of formula (III) is prepared by coupling a compound of the following formula (V) with a compound of the following formula (VI)

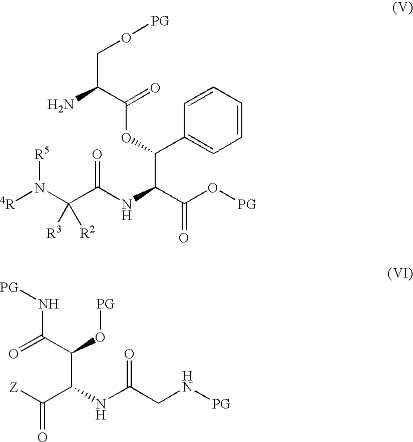

in which R² to R⁵ are as defined above,

in which Z is OH, an active ester, a pseudohalogen or a halogen, and

in which PG is H or a suitable protecting group,

and, where appropriate, partial or complete deprotection of the intermediate.

Compound (V) is thereby in particular a compound of the following formula (Va)

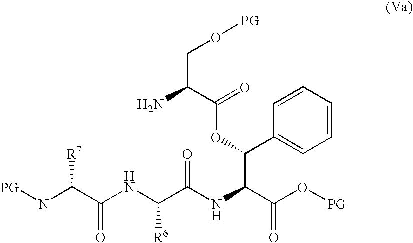

in which R⁶ and R⁷ are as defined above, and

in which PG is H or a suitable protecting group.

The coupling can take place under the same or different conditions as described above for the aforementioned coupling or the intramolecular cyclization. The coupling thereby takes place by the nucleophilic attack depicted below.

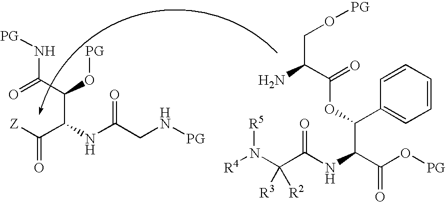

The protecting groups present in the intermediate may correspond partly, completely or not at all to those of the desired product. These can be attached, removed or replaced where appropriate by methods known to a man of the art.

The invention further relates to a compound of the following formula (III)

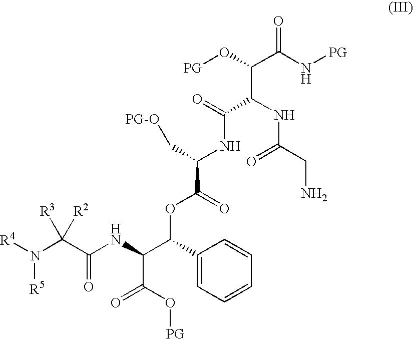

in which R² to R⁵ are as defined above, and

in which PG is H or a suitable protecting group,

in particular a compound of the following formula (IIa)

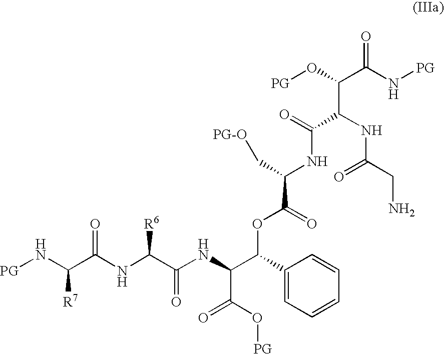

in which R⁶ and R⁷ are as defined above, and

in which PG is H or a suitable protecting group,

and especially a compound of the following formula (IIIb).

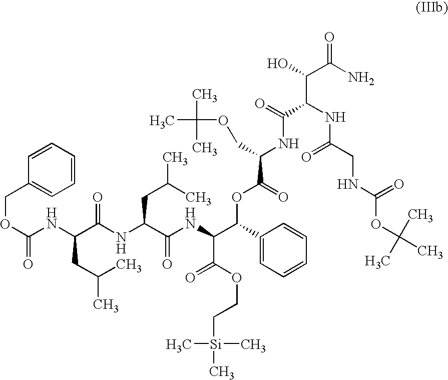

The invention further relates to a method for preparing a compound of formula (III) by coupling a compound of formula (V) with a compound of formula (VI) and, where appropriate, partial or complete deprotection of the intermediate.

The invention further relates to a compound of the following formula (VI)

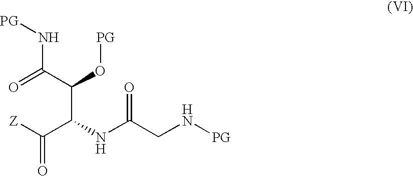

in which Z is OH, an active ester, a pseudohalogen or a halogen, and

in which PG is H or a suitable protecting group,

in particular a compound of the following formula (VIa).

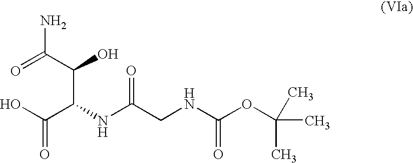

As well as a method for preparing a compound of formula (VI) by coupling a compound of the following formula (VII) with a compound of the following formula (VIII)

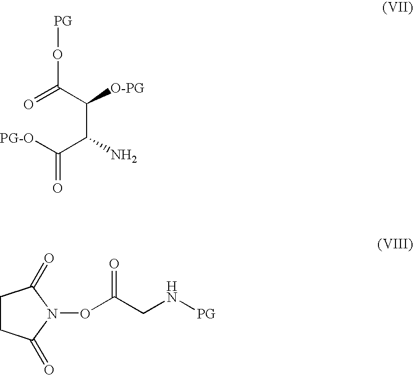

in which PG is H or a suitable protecting group,
and, where appropriate, complete or partial deprotection of the intermediate.

The compounds of formulae (VII) and (VIII) thereby are in particular compounds of the following formulae (VIIa) and (VIIIa), respectively.

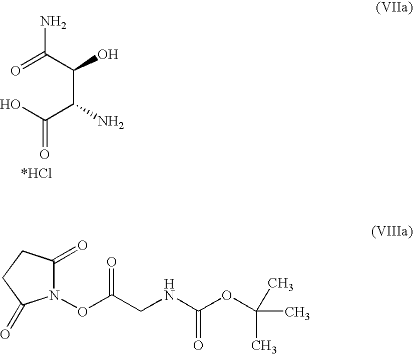

The preparation methods of the invention and the preparation of the compounds of the invention are explained in more detail by the following synthesis schemes relating to the synthesis of lysobactin.

The method is based on a modular construction of various fragments which are then combined to give a compound of formula (II). The compound of formula (II) is then subjected to an intramolecular cyclization and, where appropriate, deprotected in order to obtain the desired final product.

It has surprisingly emerged in this connection that, influenced by the choice of the building blocks and the sequence in which these are coupled, the open-chain molecules of formula (II) already having an ester linkage in their chain can be prepared. It has further been found that these open-chain compounds can be cyclized in the presence of the ester linkage to give the desired cyclic depsipeptides.

According to synthesis scheme 1, a fragment 1 is synthesized starting from (2S,3S)-2-amino-3-hydroxy-4-methoxy-4-oxobutyric acid and Boc-glycine N-hydroxysuccinimide ester.

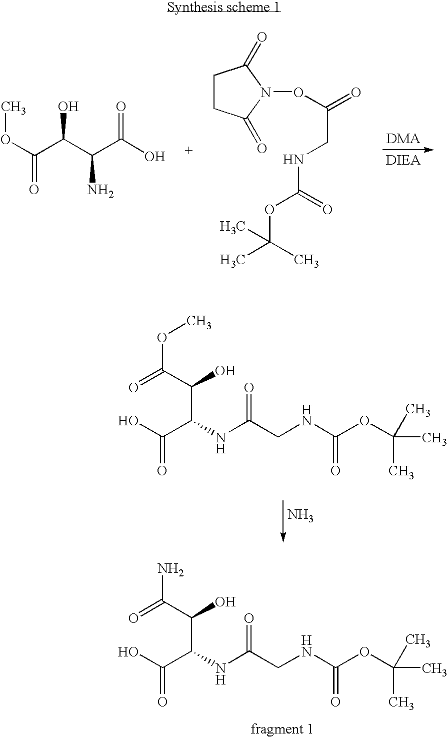

A fragment 2 is prepared according to synthesis scheme 2 starting from 3-hydroxyphenylalanine.

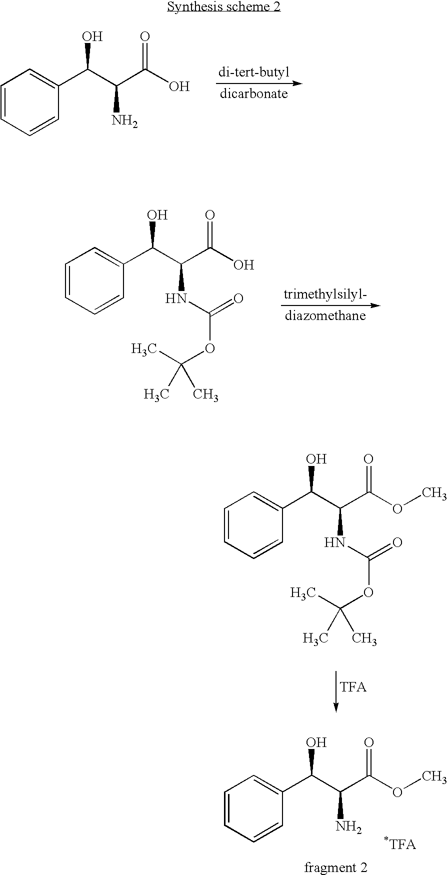

A fragment 3 is prepared according to synthesis scheme 3 starting from N²-(benzyloxycarbonyl)-D-leucine and methyl L-leucinate according to synthesis scheme 3. Compounds of formula (I) with any R² to R⁵ radicals can be prepared by replacing the two leucine derivatives in this step.

Fragments 2 and 3 are then coupled according to synthesis scheme 4, partly deprotected and the resulting intermediate is reacted with N²-(tert-butoxycarbonyl)-O³-tert-butyl-L-serine in order to obtain after a further deprotection a partly deprotected fragment 4.

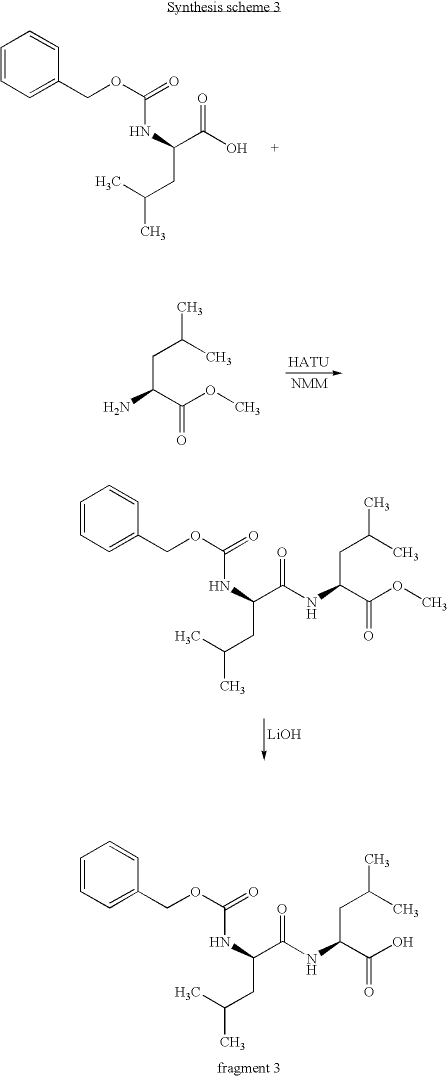

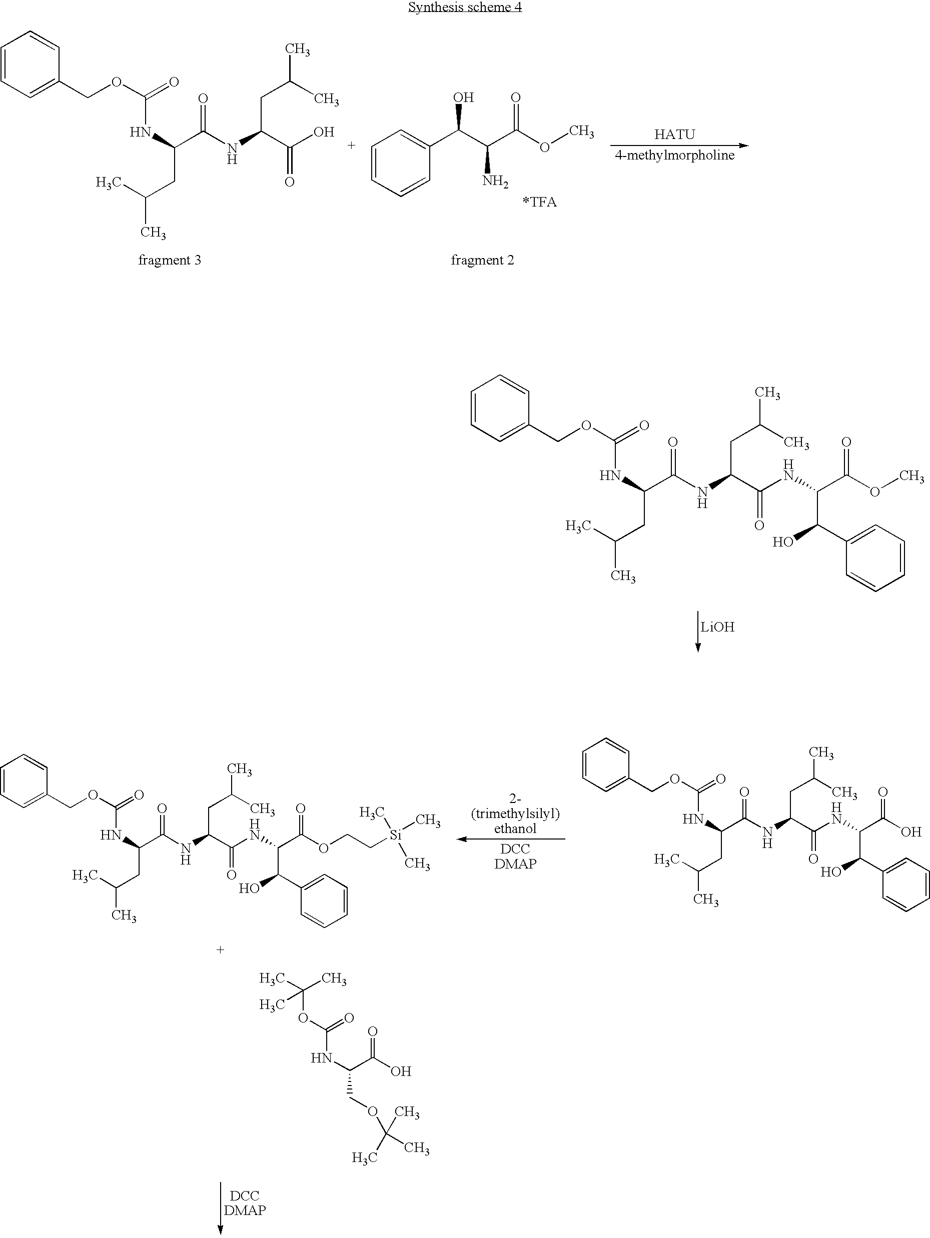

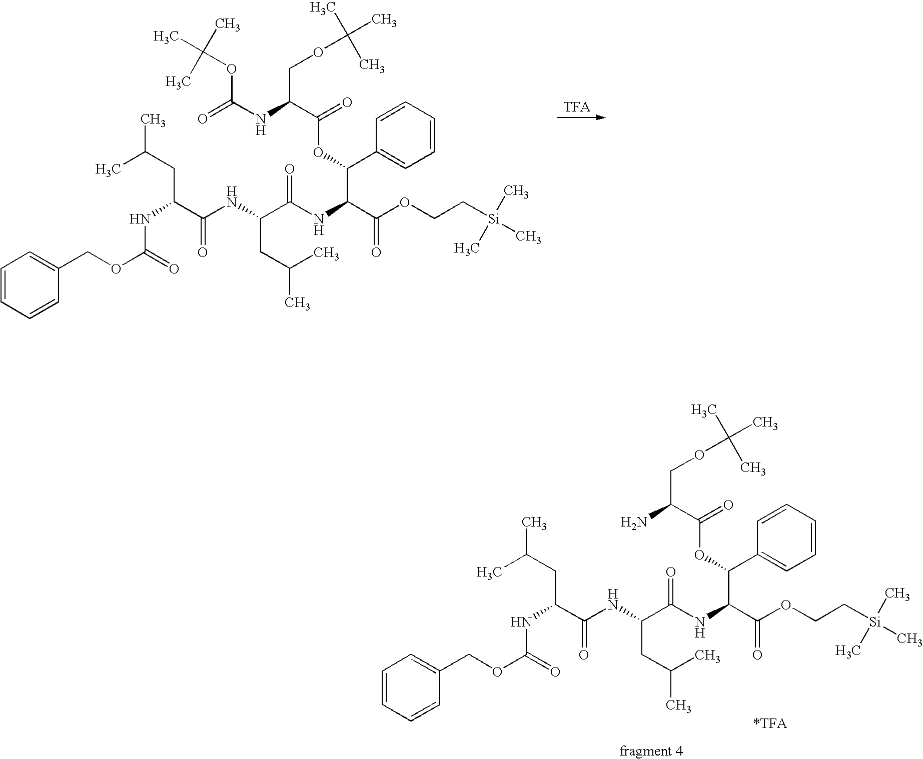

Fragment 4 obtained in this way is coupled with fragment 1 according to synthesis scheme 5 in order to obtain after partial deprotection a fragment 5.

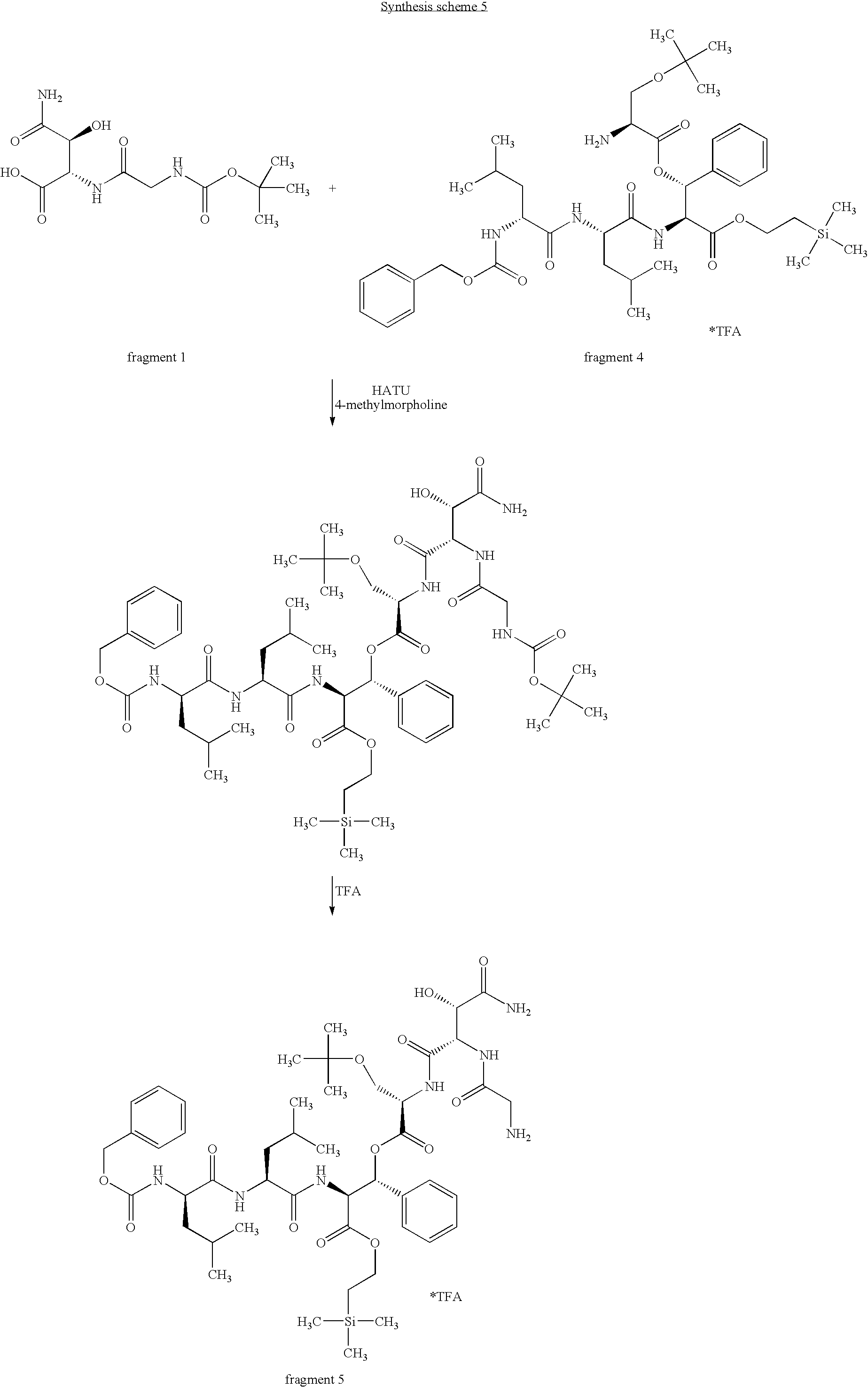

Fragment 5 is then coupled with a fragment 6 according to synthesis scheme 6. This fragment 6 is a pentapeptide which can be prepared by known methods. Instead of lysobactin, it is possible to prepare katanosin A by replacing the leucine in position 2 in fragment 6 with a valine. Deprotection of the resulting intermediate results in a fragment 7 which represents a compound of formula (II). An intramolecular cyclization and subsequent deprotection according to synthesis scheme 7 results in the desired cyclic depsipeptide, in this case lysobactin.

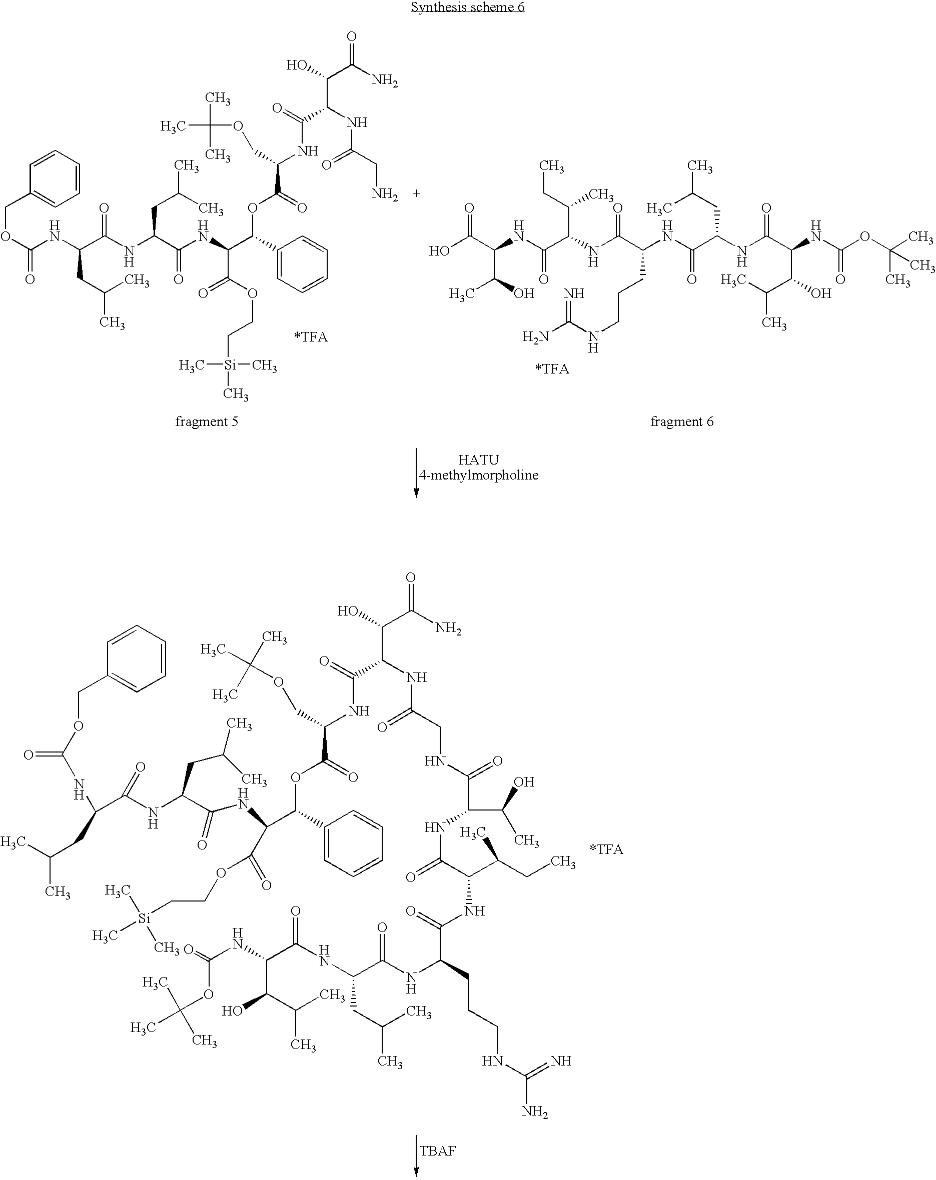

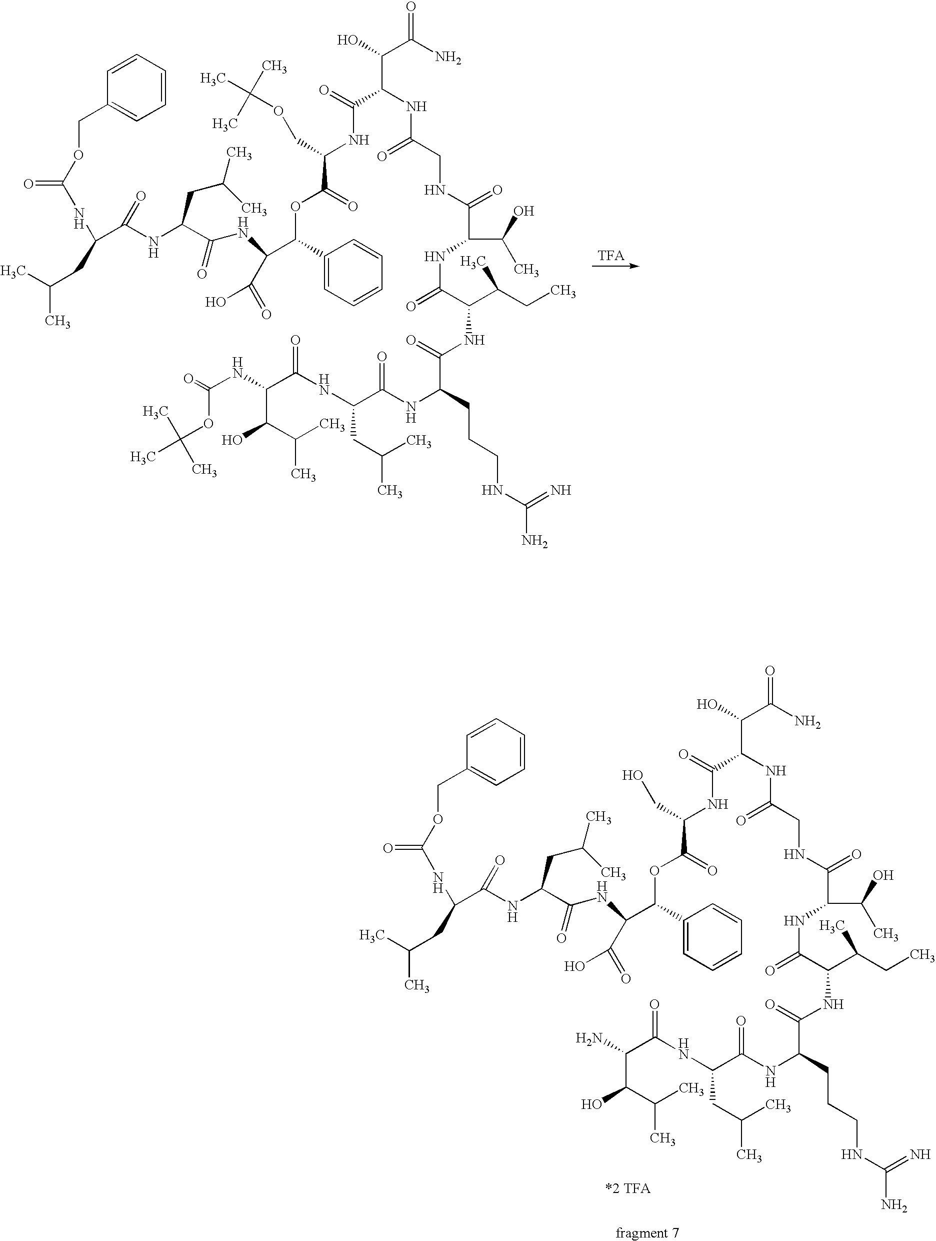

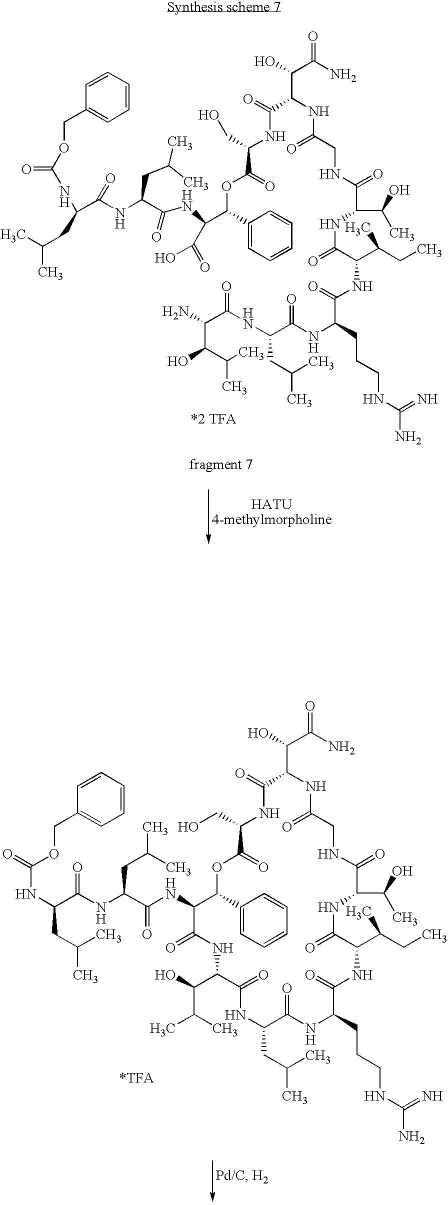

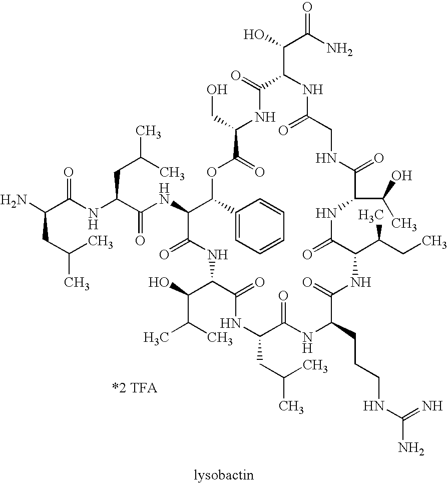

### DETAILED DESCRIPTION OF PREFERRED EMBODIMENTS

### Example

### De Novo Synthesis of Lysobactin

### ABBREVIATIONS

abs. absolute
aq. aqueous
Boc N-tert-butoxycarbonyl
conc. concentrated
DCC dicyclohexylcarbodiimide
DIEA N,N-diisopropylethylamine
DMAP 4-dimethylaminopyridine
DMF N,N-dimethylformamide
EDC 1-(3-dimethylaminopropyl)-3-ethylcarbodiimide hydrochloride
EDTA ethylenediaminetetraacetic acid
eq. equivalent(s)
fmoc 9-fluorenylmethoxycarbonyl
h hour(s)
HATU O-(7-azabenzotriazol-1-yl)-N,N,N′,N′-tetramethyluronium hexafluorophosphate
HOBT 1-hydroxy-1H-benzotriazole hydrate
LHMDS lithium hexamethyldisilazide
min minute(s)
MTBE methyl tert-butyl ether
NMM N-methylmorpholine
org. organic
RT room temperature
sat. saturated
TBAF tetrabutylammonium fluoride
TBTU N-[(1H-1,2,3-benzotriazol-1-yloxy)(dimethylamino)methylene]-N-methylmethanaminium tetrafluoroborate
TFA trifluoroacetic acid
THF tetrahydrofuran
TMG N,N,N,N-tetramethylguanidine
XPHOS dicyclohexyl(2′,4′,6′-triisopropylbiphenyl-2-yl)phosphine

### Material and Methods

### Analytical Methods—HPLC/UV

### Method 1

HPLC instrument type: HP 1100 Series; UV DAD; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min. 2 ml/min; oven: 50° C.; UV detection: 210 nm.

### Method 2

Instrument: HP 1100 with DAD detection; column: Kromasil 100 RP-18, 60 mm×2.1 mm, 3.5 μm; eluent: A=5 ml of HClO₄ (70%)/l of H₂O, B=ACN; gradient: 0 min 2% B, 0.5 min 2% B, 4.5 min 90% B, 9 min 90% B, 9.2 min. 2% B, 10 min 2% B; flow rate: 0.75 ml/min; column temp.: 30° C.; detection: UV 210 nm.

### Method 3

Instrument: Agilent 1100 with DAD (G1315B), binary pump (G1312A), autosampler (G1313A), solvent degasser (G1379A) and column thermostat (G1316A); column: Agilent Zorbax Eclipse XDB-C8 4.6×150×5 mm; column temperature: 30° C.; eluent A: 0.05% 70% perchloric acid in water; eluent B: acetonitrile; flow rate: 2.00 ml/min; gradient: 0-1 min 10% B, ramp, 4-5 min 90% B, ramp, 5.5 min 10% B.

### Method 4

HPLC instrument type: HP 1050 Series; UV DAD; column: Zorbax 300 mSB-C18 3.5μ, 4.6 mm×150 mm; eluent A: 1 l of water+0.1% TFA, eluent B: 60% acetonitrile in water with 0.1% TFA; gradient: 0.0 min 10% B, ramp, 18.0 min 80% B, 20.0 min 100% B, 25.0 min 100% B. Flow rate: 1 ml/min; oven: 40° C.; UV detection: 210 nm.

### Method 5

HPLC instrument type: HP 1050 Series; UV DAD; column: Zorbax 300 mSB-C18 3.5μ, 4.6 mm×150 mm; eluent A: 1 l of water+0.1% TFA, eluent B: 60% acetonitrile in water with 0.1% TFA; gradient: 0.0 min 10% B, 2.00 min 10% B, ramp, 50.0 min 80% B, 52.0 min 100% B, 55 min 100% B. Flow rate: 0.7 ml/min; oven: 40° C.; UV detection: 210 nm.

### Method 6

Agilent 1100 with DAD (G1315B), binary pump (G1312A), autosampler (G1313A), degasser (G1379A) and column thermostat (G1316A); column: Phenomenex Gemini 5μ C-18, 50×2 mm; oven temperature: 40° C.; eluent A: water+0.1% formic acid; eluent B: acetonitrile; flow rate: 2.00 ml/min; gradient: 0-1 min 0% B, ramp, 0-5 min 100% B, 5.50 min 100% B.

### Method 7

Daicel Chiralpak AD-H 5 μm 250 mm×2.0 mm, n-heptane/ethanol 95+5, flow rate: 0.2 ml/min, UV detection at 220 nm.

### Analytical Methods—HPLC/MS, MALDI, HR-MS

### Method 8

MS instrument type: Micromass ZQ; HPLC instrument type: Waters Alliance 2795/HP 1100; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 9

MS instrument type: Micromass ZQ; HPLC instrument type: Waters Alliance 2795/HP 1100; column: Phenomenex Gemini 3 μC-18 100 Å, 30 mm×3 mm; eluent A: 1 l of water+0.5 ml of 50% formic acid, eluent B: 111 of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 10

UV detection: 210 nm. MS instrument type: Micromass ZQ; HPLC instrument type: Waters Alliance 2795; column: Phenomenex Synergi 2p Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 11

Instrument: Micromass Platform LCZ with HPLC Agilent Series 1100; column: Thermo Hypersil GOLD 3μ 20 mm×4 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 12

Instrument: Micromass Platform LCZ with HPLC Agilent Series 1100; column: Thermo HyPURITY Aquastar 3μ 50 mm×2.1 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 13

Instrument: Micromass LCT; ionization: ESI positive/negative; HP1100 with DAD and autosampler; oven 40° C.; column: Waters Symmetry C-18, 50×2.1 mm, 3.5 μm; eluent A: 0.1% formic acid/acetonitrile, eluent B: 0.1% formic acid/water; flow rate: 0.5 ml/min; gradient: 0-1 min 0% A, 1-6 min 90% A, 6-8 min 100% A, 8-10 min 100% A, 10-15 0% A.

### Method 14

TOF-HR-MS-ESI+ spectra are recorded with a Micromass LCT instrument (capillary voltage: 3.2 KV, cone voltage: 42 V, source temperature: 120° C., desolvation temperature: 280° C.). A syringe pump (Harvard Apparatus) is used for sample delivery for this purpose. Leucine-encephalin (Tyr-Gly-Gly-Phe-Leu) is used as standard.

### Preparative Separation Methods—Hplc, Gel Chromatography

### Method 15

Instrument: Gilson Abimed HPLC; binary pump system; column: Nucleodur C₁₈ Gravity, Macherey-Nagel, 5 μm; 250×21 mm; eluent A: water/0.05%-0.1% TFA, eluent B: acetonitrile; gradient: 0-8 min 5% B, 8-40 min 5-60% B, 40-60 min 60% B, 60-75 min 60-100% B, 75-80 min 100% B, and then regeneration of the chromatography column; flow rate: 7-15 ml/min; UV detector 210 nm.

### Method 16

Instrument: Gilson Abimed HPLC; binary pump system; column: Kromasil-100A C₁₈, 5 μm; 250×30 mm; eluent A: water/0.05-0.5% TFA, eluent B: acetonitrile; gradient: 0-5 min 5% B, 5.01-10 min 10% B, 10.01-20 min 40% B, 20.01-27 min 50% B, 27.01-40 min 60% B, 40.01-45 min 90% B, 45.01-60 min 100% B; flow rate: 15-60 ml/min; UV detector 210 nm.

### Method 17

Gilson Abimed HPLC; UV detector 210 nm; column: Kromasil RP-18 5 μm, 100 Å, 250×20 mm; eluent A: water+0.05% TFA, eluent B: acetonitrile+0.05% TFA: flow rate: 10 ml/min; 0-3 min 5% B, ramp, 35 min 90% B.

### Method 18

Gilson Abimed HPLC; UV detector 210 nm; column: Gromsil ODS-4HE 10 μm, 250×40 mm; eluent A: water+0.05% TFA, eluent B: acetonitrile+0.05% TFA: flow rate: 20 ml/min; 0-3 min 10% B, ramp, 30-35 min 90% B, 35-40 min 90% B.

### Method 19

Gilson Abimed HPLC; UV detector 210 nm; column: Waters Symmetry-Prep™ C-18, 7 μm, 300×19 mm; eluent A: water+0.05% TFA, eluent B: acetonitrile+0.05% TFA: flow rate: 10 ml/min; 0-3 min 10% B, ramp, 30-38 min 90% B, 38-45 min 10% B.

### Method 20

Gel chromatography is carried out on Sephadex LH-20 (Pharmacia) without pressure. Fractions are taken (ISCO Foxy 200 fraction collector) according to UV activity (UV detector for 254 nm, Knauer). Column dimensions: 32×7 cm (1000-100 μmol scale); 30×4 cm (100-10 μmol scale); 25×2 cm (10⁻¹ μmol scale). Methanol is used as eluent.

### Method 21

Gilson Abimed HPLC; UV detector 210 nm; column: Biotage Flash40 RP-18 or compatible module Varian Metaflash C18 40M, 35 μm, 150×40 mm; eluent A: water+0.05% TFA, eluent B: acetonitrile+0.05% TFA: flow rate: 40 ml/min; 0-3 min 10% B, ramp, 30-38 min 90% B, 38-45 min 10% B.

### General Working Methods

### Procedure 1

The starting material is taken up in 30% TFA (solution in dichloromethane) and stirred at room temp. for 30 min. The solvent is then distilled out in vacuo, during which the bath temperature should not exceed 30° C. The product is then dried to constant weight under oil pump vacuum.

### A. Preparation of the Compounds

### Exemplary Compound 1A: N²-(tert-butoxycarbonyl)-L-allothreonine

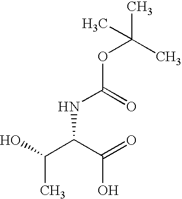

L-allo-Threonine (3.15 g, 26.44 mmol) is dissolved in water-dioxane (1+2, 75 ml), di-tert-butyl dicarbonate (6.35 g, 29.09 mmol, 1.1 equivalents) and triethylamine (4.79 ml, 34.38 mmol, 1.3 equivalents) are added, and the mixture is stirred at room temperature overnight. The solvent is then removed in vacuo. The residue is taken up in ethyl acetate and extracted with 1 M citric acid. The aqueous phase is extracted several more times with ethyl acetate until product is no longer detectable therein (HPLC, method 3). The combined organic extracts are then dried over sodium sulfate, concentrated and dried to constant weight under oil pump vacuum. The product is reacted further without further purification. Yield: 6.5 g of crude product.

HPLC (method 3): Rₜ=3.23 min. LC-MS (method 11): Rₜ=2.51 min, MS (ESIneg): m/z (%)=217.8 (100) [M−H]⁻.

¹H NMR (400 MHz, d₆-DMSO) δ (ppm)=1.08 (d, J=5.4 Hz, 3H), 1.38 (s, 9H), 3.72-3.84 (m, 2H), 6.77 (d, J=7.4 Hz, 1H).

### Exemplary Compound 2A: Benzyl N²-(tert-butoxycarbonyl)-L-allothreoninate

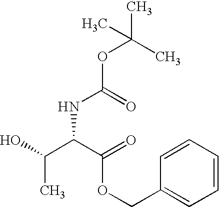

The method was carried out in analogy to the following literature: S. B. Cohen, R. Halcomb, J. Am. Chem. Soc 2004, 124, 2534-2543. W. Jiang, J. Wanner, R. J. Lee, P.-Y. Bounaud, D. L. Boger, J. Am. Chem. Soc 2003, 125, 1877-1887.

Exemplary compound 1A (6.8 g of crude product, 26.44 mmol) is taken up in methanol (177 ml), and cesium carbonate (5.56 g, 17.06 mmol, 0.63 equivalents) is added and the mixture is stirred until dissolution is complete. The solvent is then removed by distillation, DMF (42 ml) and then benzyl bromide (4.06 ml, 34.12 mmol, 1.26 equivalents) are added. The mixture is left to stir for 16 h and then most of the DMF is removed in vacuo. The residue is taken up in water and extracted with 3 portions of dichloromethane. The combined org. phases are dried over sodium sulfate, filtered and concentrated in vacuo. The crude product is purified on Biotage RP18-Flash (water-acetonitrile gradient: 0-5 min 10% ACN, 3-30 min 10-90% ACN, 30-35 min 90% ACN; flow rate: 20 ml/min). Product-containing fractions are combined and lyophilized. Yield: 5.00 g (16.16 mmol, 52% of theory) of the title compound.

HPLC (method 3): Rₜ=4.36 min.

LC-MS (method 8): Rₜ=2.39 min, MS (ESIpos): m/z (%)=332.6 (25) [M+H]⁺.

¹H NMR (400 MHz, d₆-DMSO) δ (ppm)=1.09 (d, J=6.4, 3H), 1.37 (s, 9H), 3.82 (m, 1H), 3.95 (dd, J=6.4, J=8.1 Hz), 4.98 (d, J=5.4 Hz, 1H), 5.09 (d, J=12.7 Hz, 1H), 5.16 (d, J=12.7 Hz, 1H), 7.10 (d, J=8.1 Hz, 1H), 7.31-7.37 (m, 5H).

### Exemplary Compound 3A: Benzyl L-allothreoninate Trifluoroacetate

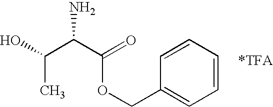

530 mg of the exemplary compound 2A are reacted according to procedure 1 with 8.0 ml of the TFA solution. The crude product (589 mg, quant.) is reacted further without further purification.

HPLC (method 3): Rₜ=3.18 min.

LC-MS (method 11): Rₜ=2.24 min, MS (ESIpos): m/z (%)=210.0 (100) [M+H]⁺.

¹H NMR (400 MHz, d₆-DMSO) δ (ppm)=1.15 (d, J=6.6 Hz, 3H), 4.09-4.10 (m, 2H), 5.26 (s, 2H), 7.36-7.44 (m, 5H), 8.34 (br. S, 2H).

### Exemplary Compound 4A: Benzyl [N²-(tert-butoxycarbonyl)-L-isoleucyl]-L-allothreoninate

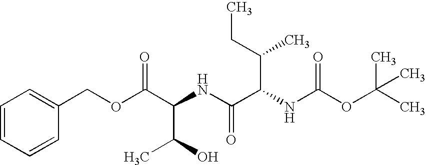

Exemplary compound 3A (2.30 g, 7.12 mmol) and N-(tert-butoxycarbonyl)-L-isoleucine (2.14 g, 9.25 mmol, 1.3 equivalents) are dissolved in DMF (21.0 ml). 4-Methylmorpholine (1.3 ml, 12.02 mmol, 1.7 equivalents) and HATU (3.52 g, 9.25 mmol, 1.3 equivalents) are added, and the mixture is stirred at room temperature for 16 h. The complete mixture is then purified by chromatography, first according to method 20 and subsequently according to method 21. Product-containing fractions are combined and lyophilized. Yield: 1.75 g (4.14 mmol, 58% of theory) as a pale beige-colored amorphous solid.

HPLC (method 3): Rₜ=4.59 min.

LC-MS (method 8): Rₜ=2.56 min, MS (ESIpos): m/z (%)=423.8 (70) [M+H]⁺.

¹H NMR (400 MHz, d₆-DMSO) δ (ppm)=0.74-0.78 (m, 6H), 1.01-1.07 (m, 2H), 1.10 (d, J=6.3 Hz, 3H), 1.37 (s, 9H), 1.64-1.66 (m, 1H), 3.86-3.94 (m, 1H), 4.28 (dd, J=7.3, J=7.3 Hz, 1H), 5.05 (d, J=5.6 Hz), 5.09 (d, J=12.7 Hz, 1H), 5.13 (d, J=12.7 Hz, 1H), 6.70 (d, J=9.0 Hz, 1H), 7.31-7.36 (m, 5H), 8.11 (d, J=8.1 Hz).

HR-TOF-MS (method 14): C₂₂H₃₅N₂O₆ calc. 423.2490, found 423.2489 [M+H]⁺.

### Exemplary Compound 5A: Benzyl L-isoleucyl-L-allothreoninate Trifluoroacetate

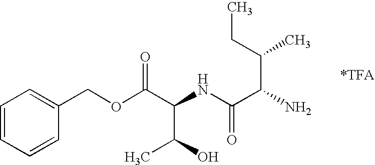

Exemplary compound 4A (224 mg, 0.53 mmol) is treated with 8.0 ml of the TFA solution according to procedure 1. 253 mg of crude product of example 5A (about 91% pure, 0.53 mmol, quant.) are obtained and are reacted without further purification.

HPLC (method 3): Rₜ=3.51 min.

LC-MS (method 8): Rₜ=1.58 min, MS (ESIpos): m/z (%) 323.6 (100) [M+H]⁺.

¹H NMR (400 MHz, d₆-DMSO) δ (ppm)=0.77-0.86 (m, 6H), 1.02 (m, 1H), 1.15 (d, J=6.4 Hz, 3H), 1.45 (m, 1H), 1.77 (m, 1H), 3.97 (m, 1H), 4.34 (m, 1H), 5.11 (d, J=12.5 Hz, 1H), 5.16 (d, J=12.5 Hz, 1H), 7.37-7.39 (m, 5H), 7.47 (m, 1H), 8.07-8.08 (m, 3H), 8.69 (d, J=7.3 Hz, 1H).

### Exemplary Compound 6A: Benzyl [N²-(tert-butoxycarbonyl)-D-arginyl]-L-isoleucyl-L-allothreoninate

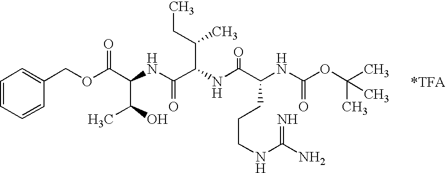

Exemplary compound 5A (253 mg 91% pure, 0.53 mmol) and N²-(tert-butoxycarbonyl)-D-arginine (145 mg, 0.53 mmol, 1 equivalent) are dissolved in DMF (3.0 ml). 4-Methylmorpholine (76 μl, 0.70 mmol, 1.3 equivalents) and HATU (221 mg, 0.58 mmol, 1.1 equivalents) are added, and the mixture is stirred at room temperature for 16 h. The complete mixture is then put onto an HPLC column and purified by chromatography (method 18). Product-containing fractions are combined and lyophilized. Yield: 364 mg (0.53 mmol, 99% of theory) of the title compound.

HPLC (method 3): Rₜ=3.91 min.

LC-MS (method 8): Rₜ=2.04 min, MS (ESIpos): m/z (%)=579.9 (100) [M+H]⁺.

¹H NMR (400 MHz, d₆-DMSO) δ (ppm)=0.72-1.16 (m, 8H), 1.37 (s, 9H), 1.46 (m, 2H), 1.60 (m, 1H), 1.69 (m, 1H), 3.06 (m, 2H), 3.93-4.01 (m, 2H), 4.25 (m, 1H), 4.33 (m, 1H), 5.07-5.14 (m, 2H), 6.96 (d, J=7.8, 1H), 7.35 (m, 5H), 7.45 (m, 1H), 7.66 (d, J=8.8), 8.33 (m, 1H).

### Exemplary Compound 7A: Benzyl D-arginyl-L-isoleucyl-L-allothreoninate Bistrifluoroacetate

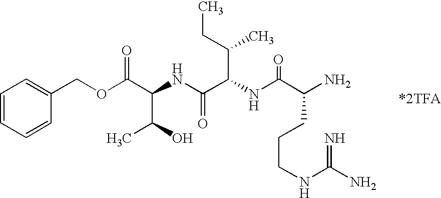

Exemplary compound 6A (237 mg, 0.34 mmol) is treated with 2.0 ml of the TFA solution according to procedure 1. 255 mg of crude product of exemplary compound 7A (94% pure, 0.34 mmol, quant.) are obtained and are reacted without further purification.

HPLC (method 3): Rₜ=3.42 min.

LC-MS (method 11): Rₜ=2.42 min, MS (ESIpos): m/z (%)=479.3 (50) [M+H]⁺.

1H NMR (400 MHz, d₆-DMSO) δ (ppm)=0.73-0.81 (m, 5H), 1.11-1.19 (m, 5H), 1.33-1.49 (m, 3H), 1.74 (m, 3H), 3.10 (m, 2H), 3.88-3.95 (m, 2H), 4.25 (dd, J=6.8, J=7.1 Hz, 1H), 4.46 (dd, J=7.3, J=8.8 Hz, 1H), 5.09 (d, J=12.5 Hz, 1H), 5.15 (dd, J=12.5 Hz), 7.36 (m, 5H), 7.61 (m, 1H), 8.10 (m, 2H), 8.51 (d, J=7.6 Hz, 1H), 8.57 (d, J=9.0 Hz, 1H).

### Exemplary Compound 8A: Benzyl [N²-(tert-butoxycarbonyl)-L-leucyl]-D-arginyl-L-isoleucyl-L-allothreoninate Trifluoroacetate

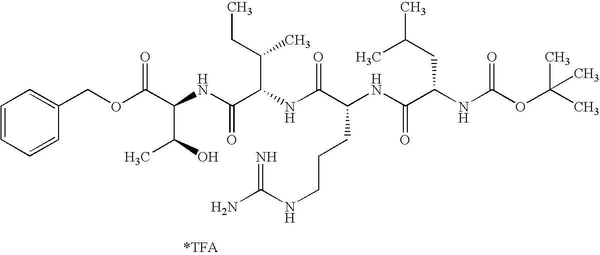

Exemplary compound 7A (240 mg, 0.34 mmol) and N-(tert-butoxycarbonyl)-L-leucine (79 mg, 0.34 mmol, 1 equivalent) are dissolved in dichloromethane-DMF (5+1, 6 ml). Diisopropylethylamine (296 μl, 1.70 mmol, 5 equivalents) and HATU (194 mg, 0.51 mmol, 1.5 equivalents) are added, and the mixture is stirred at room temperature for 24 h. The complete mixture is then put onto a gel chromatography column and purified by chromatography (method 20, eluent is methanol). Product-containing fractions are combined and concentrated. Yield: 146 mg (0.18 mmol, 53% of theory) of the title compound.

HPLC (method 3): Rₜ=4.15 min.

LC-MS (method 8): Rₜ=1.92 min, MS (ESIpos): m/z (%)=692.8 (100), [M+H]⁺.

¹H NMR (400 MHz, d₆-DMSO) δ (ppm)=0.72-1.23 (m, 22H), 1.37 (s, 9H), 1.38-1.71 (m, 3H), 3.08 (m, 2H), 3.91-4.00 (m, 2H), 4.26 (m, 1H), 4.33-4.42 (m, 2H), 5.07-5.15 (m, 2H), 6.92 (d, J=7.8 Hz, 1H), 7.35 (m, 5H), 7.47 (m, 1H), 7.88 (d, J=8.1 Hz, 1H), 7.93 (d, J=9.0 Hz, 1H), 8.35 (d, J=7.3 Hz, 1H).

### Exemplary Compound 9A: Benzyl L-leucyl-D-arginyl-L-isoleucyl-L-allothreoninate Bistrifluoroacetate

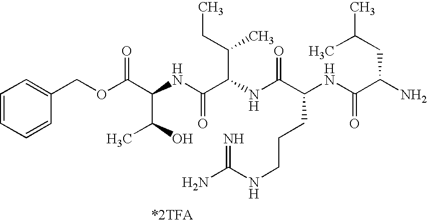

Exemplary compound 8A (220 mg, 0.27 mmol) is treated with 2.0 ml of the TFA solution according to procedure 1. 223 mg of crude product of example 9A (0.27 mmol, quant.) are obtained and are reacted without further purification.

HPLC (method 2): Rₜ=3.80.

LC-MS (method 11): Rₜ=2.54 min, MS (ESIpos): m/z (%)=592.4 (2) [M+H]⁺.

¹H NMR (400 MHz, d₆-DMSO) δ (ppm)=0.73-1.11 (m, 13H), 1.22-1.74 (m, 12H), 3.11 (m, 4H), 3.60 (m, 2H), 3.87 (m, 1H), 3.95 (m, 1H), 4.25 (m, 1H), 4.38 (dd, J=7.8, J=8.6 Hz, 1H), 4.64 (dd, J=7.8, J=13.7 Hz, 1H), 5.09 (d, J=12.7 Hz, 1H), 5.13 (d, J=12.7 Hz, 1H), 7.35 (m, 5H), 7.58 (m, 1H), 8.07 (m, 2H), 8.25 (d, J=8.8 Hz, 1H), 8.39 (d, J=7.6 Hz, 1H), 8.77 (d, J=8.3 Hz, 1H).

### Exemplary Compound 10A: Benzyl [(3R)—N²-(tert-butoxycarbonyl)-3-hydroxy-L-leucyl]-L-leucyl-D-arginyl-L-isoleucyl-L-allothreoninate Trifluoroacetate

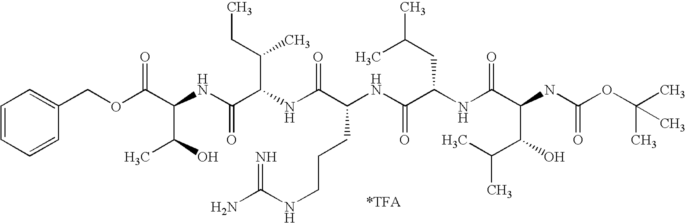

Exemplary compound 9A (223 mg, 0.27 mmol) and N-(tert-butoxycarbonyl)-(3R)-3-hydroxy-L-leucine (89 mg, 0.33 mmol, 1.22 equivalents) are dissolved in DMF (6 ml), and the solution is cooled to −20° C. 4-Methylmorpholine (150 μl, 1.36 mmol, 5 equivalents) and HATU (165 mg, 0.44 mmol, 1.6 equivalents) are added, and the mixture is stirred at room temperature for 16 h. The complete mixture is then put onto a gel chromatography column and purified by chromatography (method 20, eluent is methanol). Product-containing fractions are combined and concentrated. Yield: 188 mg (0.20 mmol, 74% of theory) of the title compound.

HPLC (method 3): Rₜ=4.24 min.

LC-MS (method 9): Rₜ=1.99 min, MS (ESIpos): m/z (%)=821.9 (100) [M+H]⁺.

¹H NMR (400 MHz, d₆-DMSO) δ (ppm)=0.71-0.90 (m, 15H), 1.00 (m, 1H), 1.10 (d, J=6.4 Hz, 3H), 1.24-1.26 (m, 3H), 1.38 (s, 9H), 1.42-1.71 (m, 6H), 3.06-3.17 (m, 3H), 3.45 (m, 1H), 3.61 (m, 1H), 3.93 (m, 1H), 4.05 (m, 1H), 4.26 (m, 1H), 4.35 (m, 2H), 4.54 (d, J=7.8 Hz, 1H), 5.07-5.15 (m, 2H), 5.45 (d, J=9.0 Hz, 1H), 7.35 (m, 5H), 7.46 (m, 1H), 7.85 (d, J=7.8 Hz, 1H), 7.89 (d, J=8.8 Hz, 1H), 7.97 (d, J=8.1 Hz, 1H), 8.35 (d, J=7.6 Hz, 1H).

### Exemplary Compound 11A: [(3R)—N²-(tert-Butoxycarbonyl)-3-hydroxy-L-leucyl]-L-leucyl-D-arginyl-L-isoleucyl-L-allothreonine Trifluoroacetate

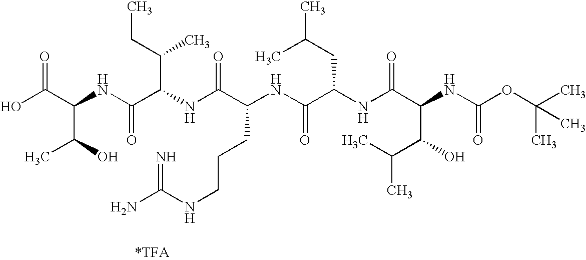

Exemplary compound 10A (100 mg, 0.11 mmol) is dissolved in glacial acetic acid (4.3 ml), 10% palladium on activated carbon (22 mg) is added, and the mixture is hydrogenated under atmospheric pressure at room temperature for 2 h. The catalyst is filtered off and the filtrate is lyophilized. The crude product is purified by chromatography (method 17). Product-containing fractions are combined and lyophilized. 58 mg (60 μmol, 55% of theory) of the title compound are obtained.

HPLC (method 3): Rₜ=3.75 min.

LC-MS (method 9): Rₜ=1.80 min, MS (ESIpos): m/z (%)=731.8 (100) [M+H]⁺.

### Exemplary Compound 12A: [N²-(tert-Butoxycarbonyl)-glycyl]-(3S)-3-hydroxy-0⁴-methyl-L-aspartic Acid

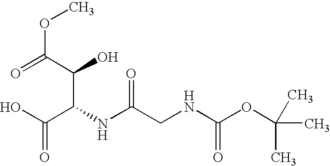

(3S)-3-Hydroxyaspartic acid is prepared according to the method of G. Cardillo, L. Gentilucci, A. Tolomelli, C. Tomasini, Synlett 1999, 1727-1730, and converted in analogy to P. G. Mattingly, M. J. Miller, J. Org. Chem. 1983, 48, 3556-3559, using microwave radiation in a closed reactor into (2S,3S)-2-amino-3-hydroxy-4-methoxy-4-oxobutyric acid hydrochloride.

(2S,3S)-2-Amino-3-hydroxy-4-methoxy-4-oxobutyric acid hydrochloride (447 mg, 2.24 mmol) are dissolved in DMF (9 ml). The solution is cooled to 0° C., Boc-glycine N-hydroxysuccinimide ester (763 mg, 2.91 mmol, 1.3 equivalents), DMAP (14 mg, 0.11 mmol, 0.05 equivalents) and finally DIEA (1170 μl, 6.72 mmol, 3 equivalents) are added. The mixture is allowed to warm slowly to room temperature and is then stirred for a further 2 h. The mixture is acidified with glacial acetic acid, mixed with acetonitrile and chromatographed on Sephadex LH 20 (method 20). Product-containing fractions are combined, concentrated and chromatographed again (method 21). Product-containing fractions are combined and lyophilized. The resulting product (761 mg, quant.) is reacted further without further purification. For analytical purposes, a pure sample is obtained by HPLC (method 19).

HPLC (method 3): Rₜ=3.15 min.

LC-MS (method 8): Rₜ=1.17 min, MS (ESIpoS)=321.2 [M+H]⁺.

[α]²⁰_{Na}=+39° (c=0.55, MeOH).

¹H NMR (300 MHz, d₆-DMSO) δ (ppm)=1.40 (s, 9H), 3.49-3.60 (m, 2H), 3.61 (s, 3H), 4.29 (m, 1H), 4.73 (d, J=6.6 Hz, 1H), 7.01 (m, 1H), 7.49 (d, J=6.99 Hz, 1H).

¹³C-NMR (d₆-acetone, 126 MHz, DEPT) δ (ppm)=28.5 (CH₃), 42.2 (CH₂), 51.8 (CH₃), 53.7 (CH), 56.0 (CH), 79.2 (quat), 169.6 (quat), 169.7 (quat), 172.8 (quat), 173.8 (quat). HR-TOF-MS (method 14): C₁₂H₂₂N₂O₈ [M+H]⁺ calc.: 321.1298, found: 321.1299.

### Exemplary Compound 13A: [N²-(tert-Butoxycarbonyl)-glycyl]-(3S)-3-hydroxy-L-asparagine

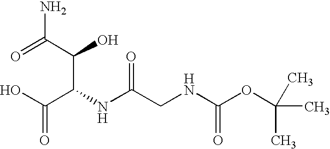

Exemplary compound 12A (353 mg, 1.10 mmol) is dissolved in 25% aqueous ammonia (1.70 ml), and the mixture is stirred at RT for about 2 h. As soon as the reaction is complete (detection by HPLC, method 3), the mixture is concentrated to dryness under oil pump vacuum, and the residue is purified by HPLC (method 17). Product-containing fractions are combined and lyophilized. Yield: 172 mg (51% of theory) of the title compound as a colorless solid.

HPLC (method 3): Rₜ=2.70 min.

LC-MS (method 11): Rₜ=2.21 min, MS (ESIpos): m/z (%)=306 (70) [M+H]⁺.

### Exemplary Compound 14A: (3R)-3-Hydroxyphenylalanine

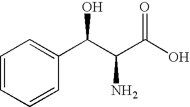

This exemplary compound is synthesized according to the method of Belokon (Y. N. Belokon, K. A. Kochetkov, N. S. Ikonnikov, T. V. Strelkova, S. R. Harutyunyan, A. S. Saghiyan, Tetrahedron: Asymmetry 2001, 12, 481-485).

LC-MS (method 12): Rₜ=0.41 min, MS (ESIpos): m/z (%)=182.1 (100) [M+H]⁺.

[α]²⁰_{Na}=−21° (c=0.1, MeOH). Lit (D. Alker, G. Hamblett, L. M. Harwood, S. M. Robertson, D. J. Watkin, C. E. Williams, Tetrahedron 1998, 54, 6089-6098): [α]²²_{Na}=−200 (c=0.8, MeOH).

¹H NMR (400 MHz, D₂O) δ (ppm)=3.84 (d, J=4.5 Hz, 1H), 4.64 (d, J=4.5 Hz, 1H), 7.30-7.36 (m, 5H).

### Exemplary Compound 15A: N-Butoxycarbonyl-(3R)-3-hydroxyphenylalanine

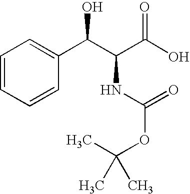

Exemplary compound 14A (0.5 g, 2.76 mmol) is taken up in 1,4-dioxane-water (2+1, 9 ml), and triethylamine (500 μl, 3.59 mmol, 1.3 equivalents) and di-tert-butyl dicarbonate (660 mg, 3.04 mmol, 1.3 equivalents) are added. The mixture is stirred at room temperature for 16 h and then stopped using 1M citric acid. The mixture is extracted with several portions of ethyl acetate until product is no longer detectable in the aqueous phase by HPLC (method 3). The combined organic phases are dried over sodium sulfate and concentrated. 759 mg (2.70 mmol, 98% of theory) of the title compound are obtained as a colorless oil in the residue.

HPLC (method 3): Rₜ=3.89 min.

LC-MS (method 8): Rₜ=1.87 min, MS (ESIpos): m/z (%)=282.3 (40) [M+H]⁺.

¹H NMR (400 MHz, d₆-DMSO) δ (ppm)=1.27 (s, 9H, COC(CH₃)₃), 4.21 (m, 1H), 5.09 (s, 1H), 6.30 (d, J=9.8 Hz), 7.22-7.34 (m, 5H).

Chiral HPLC (method 7): e.e. 94.1%.

### Exemplary Compound 16A: Methyl N-butoxycarbonyl-(3R)-3-hydroxyphenylalaninate

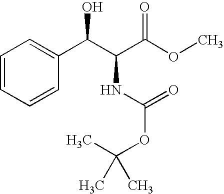

Exemplary compound 16A (331 mg, 1.11 mmol) is dissolved in dichloromethane-methanol (5+1, 12 ml), cooled to 0° C., and trimethylsilyldiazomethane (2M in THF, 1.66 ml, 3.32 mmol, 3 equivalents) is added dropwise. The mixture is stirred at 0° C. for a further 30 min and then a few drops of TFA are added until decolorization occurs. The solvent is distilled off, and as residue remains the title compound (345 mg, 95% pure according to HPLC) in quantitative yield as a yellowish oil.

HPLC (method 3): Rₜ=4.26 min.

LC-MS (method 8): Rₜ=2.11 min, MS (ESIpos): m/z (%)=296.3 (50) [M+H]⁺.

¹H NMR (300 MHz, d₆-DMSO) δ (ppm)=1.27 (s, 9H, COC(CH₃)₃), 3.60 (s, 3H, OCH₃), 4.31 (dd, J=3.8, J=9.3 Hz, 1H), 5.03 (d, J=3.4 Hz, 1H), 5.68 (br s, 1H), 6.62 (d, J=9.1 Hz, 1H), 7.23-7.34 (m, 5H).

### Exemplary Compound 17A: Methyl (3R)-3-hydroxy-phenylalaninate Trifluoroacetate

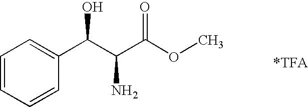

Exemplary compound 16A (345 mg, 1.17 mmol) is dissolved in 30% TFA in dichloromethane (10 ml) and stirred at RT for 15 min. The solvent is then distilled off. The residue is dried to constant weight under oil pump vacuum. Yield: 401 mg (quant.) as a yellow oil which is employed without further purification in the next step.

HPLC (method 3): Rₜ=2.51 min.

LC-MS (method 8): Rₜ=0.30 min, MS (ESIpos): m/z (%)=196.1 (20) [M+H]⁺.

### Exemplary Compound 18A: Methyl [N²-(benzyloxycarbonyl)-D-leucyl]-L-leucinate

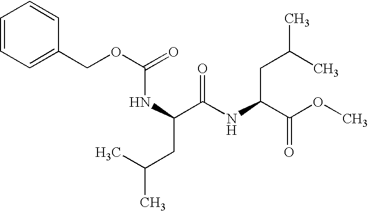

N²-(Benzyloxycarbonyl)-D-leucine (BACHEM Cat No z13351.) (6.37 g, 24 mmol) and methyl L-leucinate (3.49 g, 24 mmol, 1 eq.) are dissolved in DMF (75 ml) at 0° C., and then NMM (5.28 ml, 48 mmol, 2 eq.) and HATU (13.69 g, 36 mmol, 1.5 eq.) are added. The mixture is stirred at room temperature for three hours. MTBE and a saturated sodium bicarbonate solution are added, and extraction is carried out. The aqueous phase is extracted again with a second portion of MTBE, and the combined organic phases are then washed with 1M citric acid and again with a saturated sodium bicarbonate solution, dried over sodium sulfate, filtered and concentrated in vacuo. The residue is purified by chromatography in two portions (Biotage 40M, cyclohexane/ethyl acetate 3+1). Product-containing fractions are combined and lyophilized. Yield: 7.85 g (80% of theory) of the title compound.

HPLC (method 3): Rₜ 4.82 min.

LCMS (method 8): Rₜ 2.65 min; MS (ESIpos.): m/z (%)=393 (100) [M+H]⁺.

[α]²⁰_{Na}=−5.2° (c=0.52, MeOH).

¹H NMR (400 MHz, d₆-DMSO) δ (ppm)=0.77-0.92 (m, 12H), 1.31-1.66 (m, 6H), 3.60 (s, 3H), 4.10 (m, 1H), 4.28 (m, 1H), 5.02 (s, 2H), 7.25-7.38 (m, 6H), 8.23 (d, 1H).

¹³C-NMR (126 MHz, d₆-DMSO) δ (ppm)=21.1 (CH₃), 21.5 (CH₃), 22.8 (CH₃), 22.9 (CH₃), 24.2 (CH), 41.0 (CH₂), 50.0 (CH), 51.8 (CH₃, OCH₃), 52.9 (CH), 65.3 (CH₂, OCH₂Ph), 127.6 (CH, ar-C), 127.7 (CH, ar-C), 128.3 (CH, ar-C), 137.1 (C quat, ar-C), 155.8 (C quat, NCOC(CH₃)₃), 172.4 (C quat, C═O), 172.9 (C quat, C═O).

### Exemplary Compound 19A: [N²-(Benzyloxycarbonyl)-D-leucyl]-L-leucine

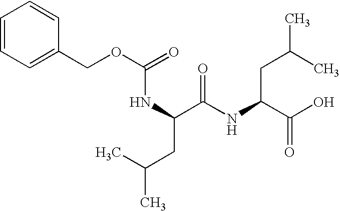

Exemplary compound 18A (7.70 g, 19.62 mmol) is taken up in 200 ml of THF/water (3+1), cooled to 0° C., and lithium hydroxide monohydrate (1.65 g, 39.24 mmol, 2 eq.) is added. The mixture is left to stir at 0° C. until according to HPLC monitoring (method 3) the reaction has proceeded to completion (about 45 min). Most of the THF is distilled off in vacuo, the mixture is adjusted to about pH 4 by adding citric acid, and the mixture is extracted with 2 portions of ethyl acetate. The combined org. phases are dried over sodium sulfate, filtered and concentrated. The product is obtained as a colorless amorphous substance in a yield of 6.87 g (89% of theory) of the title compound.

HPLC (method 3): Rₜ=4.45 min.

LC-MS (method 8): Rₜ=2.39 min, MS (ESIpos) m/z (%)=379 (100) [M+H]⁺, 757 (40) [2M+H]⁺.

[α]²⁰_{Na}=+4.7° (c=0.50, MeOH).

¹H NMR (300 MHz, d₆-DMSO) δ (ppm)=0.77-0.92 (m, 12H), 1.34-1.68 (m, 6H), 4.04-4.26 (m, 2H), 5.02 (s, 2H), 7.25-7.38 (m, 6H), 8.12 (d, 1H), 12.50 (br. s, 1H).

HR-TOF-MS (method 14): C₂₀H₃₁N₂O₅ [M+H]⁺ calc. 379.2228, found 379.2216.

### Exemplary Compound 20A: Methyl [N²-(benzyloxycarbonyl)-D-leucyl]-L-leucyl-(3R)-3-hydroxyphenylalaninate

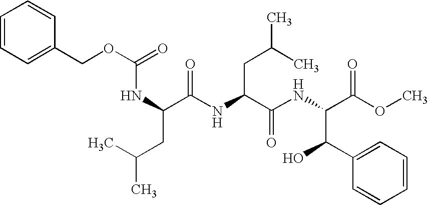

Exemplary compound 19A (550 mg, 1.45 mmol) and exemplary compound 17A (449 mg, 1.45 mmol, 1 equivalent) are dissolved in DMF (12 ml) at 0° C. 4-methylmorpholine (320 μl, 2.9 mmol, 2 equivalents) and HATU (663 mg, 1.74 mmol, 1.2 equivalents) are then added, and the mixture is stirred at 0° C. for 15 min. Subsequently further 4-methylmorpholine (160 μl, 1.45 mmol, 1 equivalent) is added, and the mixture is stirred at RT for 16 h. The mixture is then extracted between ethyl acetate and conc. sodium bicarbonate, and the organic phase is washed with 0.5M citric acid and again with conc. sodium bicarbonate, dried over sodium sulfate and concentrated. The residue is purified by chromatography (method 17). Product-containing fractions are combined and lyophilized. Yield: 626 mg (1.13 mmol, 78% of theory) of the title compound.

HPLC (method 3): Rₜ=4.69 min.

LC-MS (method 8): Rₜ=2.58 min, MS (ESIpos): m/z (%) 556.5 (100) [M+H]⁺.

¹H NMR (300 MHz, d₆-DMSO) δ (ppm)=0.76-0.88 (m, 12H), 1.23-1.60 (m, 6H), 3.54 (s, 3H, OCH₃), 4.06-4.11 (m, 1H), 4.43 (dd, J=8.3, J=14.9 Hz, 1H), 4.52 (dd, J=4.1, J=7.7 Hz, 1H), 5.02-5.06 (m, 3H), 5.87 (d, J=4.5 Hz, 1H), 7.20-7.40 (m, 11H), 8.01 (d, J=8.7 Hz, 1H), 8.08 (d, J=8.5 Hz, 1H).

### Exemplary Compound 21A: [N²-(Benzyloxy)carbonyl-D-leucyl]-L-leucyl-(3R)-3-hydroxyphenylalanine

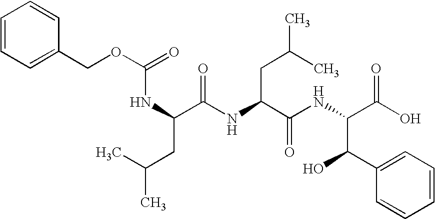

Exemplary compound 20A (650 mg, 1.17 mmol) is dissolved under argon in THF-water (2+1, 30 ml). At 0° C., an aqueous solution of lithium hydroxide (57 mg, 2.40 mmol, 4 equivalents in 8.65 ml of water) is added dropwise. The reaction has proceeded to completion after 45 min (HPLC, method 1). Glacial acetic acid is added, and the mixture is concentrated. The crude product is purified by chromatography (method 16). Product-containing fractions are combined and lyophilized. Yield: 618 mg (98% of theory) of the title compound.

HPLC (method 1): Rₜ=2.44 min.

LC-MS (method 13) Rₜ=6.04; MS (ESIpos) m/z (%)=542.5 (100) [M+H]⁺, 183.8 (80) [2M+H]⁺; MS (ESIneg) m/z (%)=540.4 (40) [M−H]⁻; 1081.70 (100) [2M−H]⁻.

### Exemplary Compound 22A: 2-(Trimethylsilyl)ethyl-N²-[benzyloxycarbonyl-D-leucyl]-L-leucyl-(3R)-3-hydroxy-L-phenylalaninate

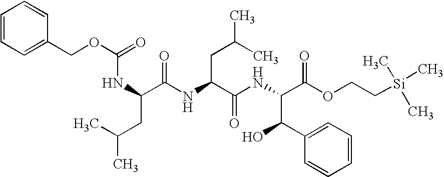

Exemplary compound 21A (150 mg, 277 μmol) and 2-(trimethylsilyl)ethanol (790 μl, 5.54 mmol, 20 equivalents) and some 4 Å molecular sieves are dissolved in dry dichloromethane (3.0 ml) and stirred at −30° C. for about 1 h. DCC (114 mg, 553 mol, 2 equivalents) and DMAP (34 mg, 277 μmol, 1 equivalent) are then added, and the mixture is stirred overnight and allowed to reach room temperature slowly during this. The mixture is then concentrated in vacuo and chromatographed (method 16). Product-containing fractions are combined and lyophilized. Yield: 108 mg (60% of theory) of the title compound.

HPLC (method 1): Rₜ=3.14 min.

LC-MS (method 10): Rₜ=2.97 min, MS (ESIpos): m/z (%)=642.3 (100) [M+H]⁺.

### Exemplary Compound 23A: 2-(Trimethylsilyl)ethyl N²-[(benzyloxy)carbonyl]-D-leucyl-L-leucyl-(3R)-3-{[N²-(tert-butoxycarbonyl)-O³-(tert-butyl)-L-seryl]oxy}-L-phenylalaninate

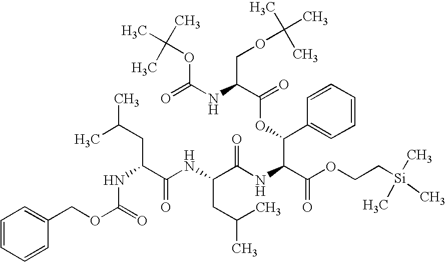

Exemplary compound 22A (104 mg, 162 μmol) and N²-(tert-butoxycarbonyl)-O³-tert-butyl-L-serine (47 mg, 178 μmol, 1.1 equivalents) are dissolved in dry dichloromethane (2.0 ml) and some 4 Å molecular sieves are added. DCC (70 mg, 340 mol, 2.1 equivalents) and DMAP (23 mg, 194 μmol, 1.2 equivalents) are then added, and the mixture is stirred overnight and allowed slowly to reach room temperature during this. The mixture is then concentrated in vacuo and chromatographed (method 16). Product-containing fractions are combined and lyophilized. Yield: 120 mg (84% of theory) of the title compound.

HPLC (method 1): Rₜ=3.49 min.

LC-MS (method 10): Rₜ=3.38 min, MS (ESIpos): m/z (%)=885.6 (100) [M+H]⁺.

HR-TOF-MS (method 14): C₄₆H₇₃N₄O₁₁Si calc. 885.5040, found 885.5031 [M+H]⁺.

### Exemplary Compound 24A: 2-(Trimethylsilyl)ethyl N²-[(benzyloxy)carbonyl]-D-leucyl-L-leucyl-(3R)-3-{[O³-(tert-butyl)-L-seryl]oxy}-L-phenylalaninate Trifluoroacetate

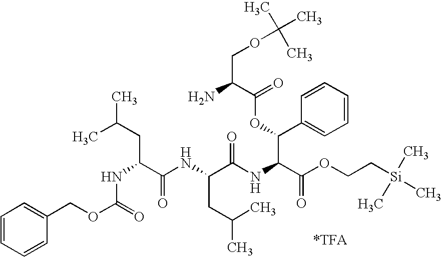

Exemplary compound 23A (117 mg, 132 μmol) is dissolved in dichloromethane (3 ml). 15% TFA in dichloromethane (20 ml) is added and, after 10 min, the mixture is concentrated to dryness. The residue is purified by chromatography (method 16). Product-containing fractions are combined and lyophilized. Yield: 100 mg (83% of theory).

HPLC (method 1): Rₜ=2.40 min.

LC-MS (method 10): Rₜ=2.28 min, MS (ESIpos): m/z (%)=785.4 (100) [M+H]⁺.

### Exemplary Compound 25A: 2-(Trimethylsilyl)ethyl N²-[(benzyloxy)carbonyl]-D-leucyl-L-leucyl-(3R)-3-{[N²-(tert-butoxycarbonyl)glycyl-(3S)-3-hydroxy-L-asparaginyl-O³-(tert-butyl)seryl]oxy}-L-phenylalaninate

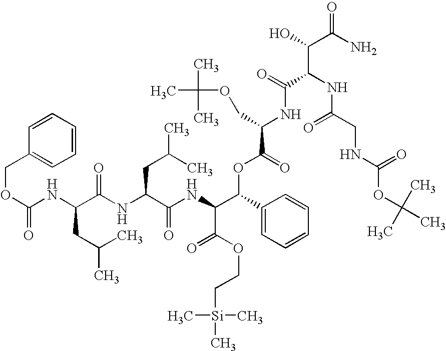

Exemplary compound 24A (96 mg, 107 μmol) and exemplary compound 13A (33 mg, 107 μmol, 1 equivalent) are dissolved in DMF (2.0 ml) and cooled to −30° C. HATU (122 mg, 320 μmol, 3 equivalents) and 4-methylmorpholine (86 mg, 854 μmol, 8 equivalents) are added, and the mixture is then allowed slowly to warm to about 4° C. and is left to stand at this temperature for 12 h. The crude reaction solution is chromatographed (method 15), and product-containing fractions are combined and lyophilized. Yield: 92 mg (89% of theory) of the title compound.

HPLC (method 1): Rₜ=3.22 min.

LC-MS (method 9): Rₜ=3.21 min, MS (ESIpos): m/z (%)=1072.6 (100) [M+H]⁺; MS (ESIneg): m/z (%)=1070.5 (100) [M−H]⁻.

HR-TOF-MS (method 14): C₅₂H₈₂N₇O₁₅Si calc. 1072.5633, found 1072.5667 [M+H]⁺.

### Exemplary Compound 26A: 2-(Trimethylsilyl)ethyl N²-[(benzyloxy)carbonyl]-D-leucyl-L-leucyl-(3R)-3-{[glycyl-(3S)-3-hydroxy-L-asparaginyl-O³-(tert-butyl)seryl]oxy}-L-phenylalaninate Trifluoroacetate

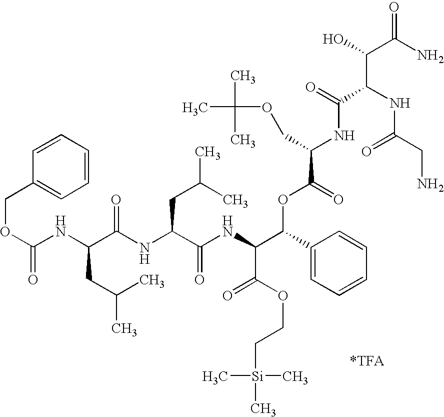

Exemplary compound 25A (90 mg, 84 μmol) is dissolved in dichloromethane (3.0 ml). 15% TFA in dichloromethane (20 ml) is added and, after 10 min, the mixture is concentrated to dryness. The residue is purified by chromatography (method 16). Product-containing fractions are combined and lyophilized. Yield: 73 mg (80% of theory) of the title compound.

HPLC (method 1): Rₜ=2.65 min.

LC-MS (method 10): Rₜ=2.13 min, MS (ESIpos): m/z (%)=972.6 (100) [M+H]⁺; MS (ESIneg): m/z (%)=970.7 (100) [M−H]⁻.

HR-TOF-MS (method 14): C₄₇H₇₄N₇O₁₃Si calc. 972.5109, found 972.5103 [M+H]⁺.

### Exemplary Compound 27A: 2-(Trimethylsilyl)ethyl N²-[(benzyloxy)carbonyl]-D-leucyl-L-leucyl-(3R)-3-{[N²-(tert-butoxycarbonyl)-(3R)-3-hydroxy-L-leucyl-L-leucyl-D-arginyl-L-isoleucyl-L-allothreonyl-glycyl-(3S)-3-hydroxy-L-asparaginyl-O³-(tert-butyl)seryl]oxy}-L-phenylalaninate trifluoroacetate

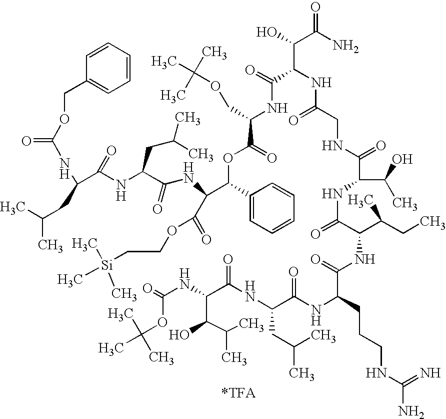

Exemplary compound 26A (10.0 mg, 9.2 μmol) and exemplary compound 11A (8.2 mg, 107 μmol, 1 equivalent) are dissolved in DMF (0.5 ml) and cooled to −30° C. HATU (10.5 mg, 27.6 μmol, 3 equivalents) and 4-methylmorpholine (7.5 mg, 74 μmol, 8 equivalents) are added, and the mixture is then slowly allowed to warm to about 4° C. and is left to stand at this temperature for 12 h. The crude reaction solution is chromatographed (method 15), and product-containing fractions are combined and lyophilized. Yield: 10.7 mg (65% of theory) of the title compound.

HPLC (method 1): Rₜ=2.99 min.

LC-MS (method 9): Rₜ=2.40 min, MS (ESIpos): m/z (%)=1685.8 (50) [M+H]⁺; MS (ESIneg): m/z (%)=1683.8 (50) [M−H]⁻, 1728.8 (100) [M+HCOOH]⁻.

HR-TOF-MS (method 14): C₈₀H₁₃₄N₁₅O₂₂Si calc. 1684.9592, found 1684.9573 [M+H]⁺.

### Exemplary Compound 28A: N²-[(Benzyloxy)carbonyl]-D-leucyl-L-leucyl-(3R)-3-{[N²-(tert-butoxycarbonyl)-(3R)-3-hydroxy-L-leucyl-L-leucyl-D-arginyl-L-isoleucyl-L-allothreonyl-glycyl-(3S)-3-hydroxy-L-asparaginyl-O³-(tert-butyl)seryl]oxy}-L-phenylalanine Trifluoroacetate

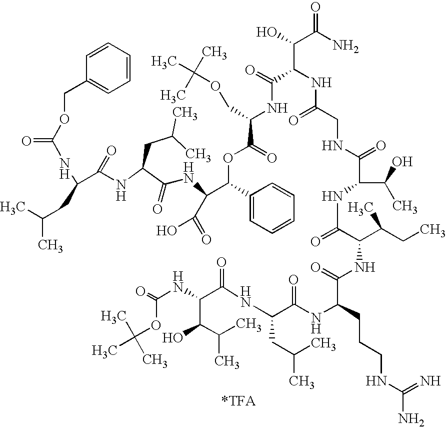

### Method A:

Exemplary compound 27A (10 mg, 5.6 μmol) is dissolved in abs. THF (0.5 ml). TBAF (17.4 mg, 67 μmol, 12 equivalents) is added, and the mixture is stirred at RT for 1 h. According to HPLC analysis (method 1), the reaction is complete, and the reaction is stopped with glacial acetic acid (6 μl), and the mixture is concentrated and chromatographed (method 15). Product-containing fractions are combined and lyophilized. Yield: 2.5 mg (about 69% pure, 18% of theory) of the title compound.

### Method B:

Compound 27A (25 mg, 13.9 μmol) is dissolved in abs. THF (2.5 ml). Sodium sulfate (anhydrous, 200 mg, 1.4 mmol) is added and the suspension is stirred for 30 min. TBAF solution (1M anhydrous in THF, 84 μl, 6 equivalents) is added, and the mixture is stirred at RT for 45 minutes. According to HPLC analysis (method 1), the reaction is complete. The reaction is stopped with glacial acetic acid (16 μl), and the mixture is filtered, concentrated and chromatographed (method 15). Yield: 21 mg (>95% pure, 89% of theory) of the title compound.

HPLC (method 6): Rₜ=2.99 min.

LC-MS (method 9): Rₜ=2.34 min, MS (ESIpos): m/z (%)=1585.6 (20) [M+H]⁺; MS (ESIneg): m/z (%)=1583.4 (100) [M−H]⁻.

HR-TOF-MS (method 14): C₇₅H₁₂₁N₁₅O₂₃ calc. 1584.8884, found 1584.8843 [M+H]⁺.

### Exemplary Compound 29A: N²-[(Benzyloxy)carbonyl]-D-leucyl-L-leucyl-(3R)-3-{[(3R)-3-hydroxy-L-leucyl-L-leucyl-D-arginyl-L-isoleucyl-L-allothreonyl-glycyl-(3S)-3-hydroxy-L-asparaginyl-seryl]oxy}-L-phenylalanine Bistrifluoroacetate

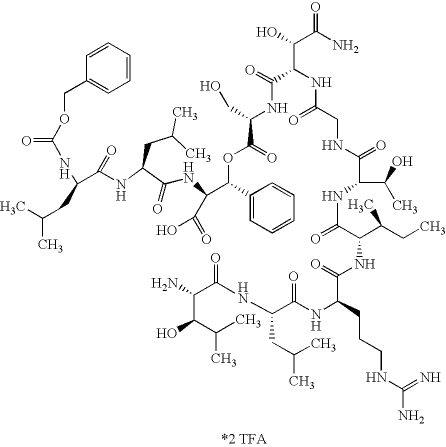

Exemplary compound 28A (2.5 mg, 1.5 μmol) is reacted with triisopropylsilane (12.5 μl) and water (2.8 μl), and 0.5 ml of TFA is added. The mixture is then stirred at room temperature for 1 h and finally the solvent is removed in vacuo. The residue is chromatographed (method 15). Product-containing fractions are combined and lyophilized. Yield: 2 mg (82% of theory) of the title compound.

HPLC (method 6): Rₜ=2.00 min.

LC-MS (method 9): Rₜ=1.60 min, MS (ESIpos): m/z (%)=714.9 (100) [M+2H]²⁺; MS (ESIneg) m/z (%)=1427.7 (100) [M−H]⁻.

HR-TOF-MS (method 14): C₆₆H₁₀₆N₁₅O₂₀ calc. 1428.7734, found 1428.7700 [M+H]⁺.

### Exemplary Compound 30A: N-Benzyloxycarbonyl-lysobactin Trifluoroacetate

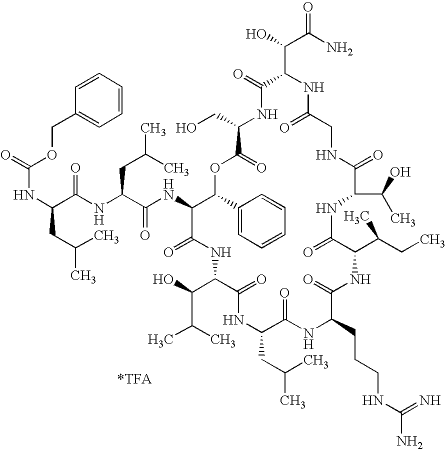

Exemplary compound 29A (1.0 mg, 1.1 μmol) is dissolved in DMF (0.9 ml) and cooled to −15° C. HATU (1.2 mg, 3.3 μmol, 3 equivalents) and 4-methylmorpholine (1 μl of a solution of 100 μl of 4-methylmorpholine in 0.9 ml of DMF, 8.7 μmol, 8 equivalents) are added, and the mixture is then slowly allowed to warm to about 4° C. and is stirred at room temperature for 3 h. The crude reaction solution is chromatographed (method 15), and product-containing fractions are combined and lyophilized. Yield: 1.2 mg (73% of theory) of the title compound.

HPLC (method 1): Rₜ=2.17 min.

LC-MS (method 9): Rₜ=2.00 min, MS (ESIpos): m/z (%)=1410.8 (100) [M+H]⁺; MS (ESIneg) m/z (%)=1408.7 (100) [M−H]⁻.

HR-TOF-MS (method 14): C₆₆H₁₀₄N₁₅O₁₉ calc. 1410.7628, found 1410.7639 [M+H]⁺.

### Exemplary Compound 31A: Lysobactin Bistrifluoroacetate

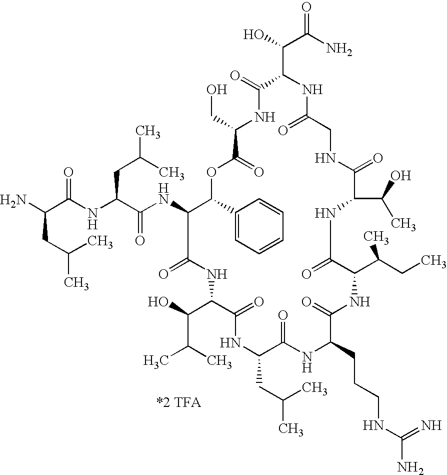

Exemplary compound 30A (1.0 mg, 0.66 μmol) is dissolved in dioxane (0.5 ml), 0.75 ml of 0.1% aq. TFA and a spatula tip of 10% Pd/C are added, and the mixture is hydrogenated under atmospheric pressure at RT for 15 min. The product is filtered to remove the catalyst, concentrated and purified by chromatography (method 15). Yield: 0.6 mg (61% of theory) of the title compound.

HPLC (method 4): Rₜ=16.31 min. The identity of the synthesized product 31A was confirmed by coinjection with authentic lysobactin (obtained by the method described in WO 2004/099239 (A1)).

HPLC (method 5) Rₜ=38.10 min. The identity of the synthesized product 31A was confirmed by coinjection with authentic lysobactin (obtained by the method described in WO 2004/099239 (A1)).

LC-MS (method 9): Rₜ=1.40 min, MS (ESIpos): m/z (%) 638.9 (100) [M+2H]²⁺, 1276.8 (5) [M+H]⁺; MS (ESIneg): m/z (%)=637.0 (100) [M−2H]²⁻, 1274.7 (40) [M−H]⁻.

HR-TOF-MS (method 14): C₅₈H₉₈N₁₅O₁₇ calc. 1276.7260, found 1276.7264 [M+H]⁺.

### B. Assessment of the Physiological Activity

The in vitro effect of the compounds of the invention can be shown in the following assay:

### Determination of the Minimum Inhibitory Concentration (MIC):

The MIC is determined in the liquid dilution test in accordance with the NCCLS guidelines. Overnight cultures of Staphylococcus aureus 133, Entercococcus faecalis ICB27159 and Streptococcus pneumoniae G9a are incubated with the described test substances in a 1:2 dilution series. The MIC determination is carried out with a cell count of 10⁵ microbes per ml in Isosensitest medium (Difco, Irvine/USA), with the exception of S. pneumoniae, which is tested in BHI broth (Difco, Irvine/USA) with 10% bovine serum at a cell count of 10⁶ microbes per ml. The cultures are incubated at 37° C. for 18-24 hours, S. pneumoniae in the presence of 10% CO₂.

The MIC is defined as the lowest concentration of each substance at which no visible bacterial growth occurs any longer. The MIC values are reported in μg/ml.

No significant differences in the physiological activity emerged between lysobactin prepared by complete synthesis and fermented lysobactin.

What is claimed is:

1. A method for preparing cyclic depsipeptides of the following formula (I) 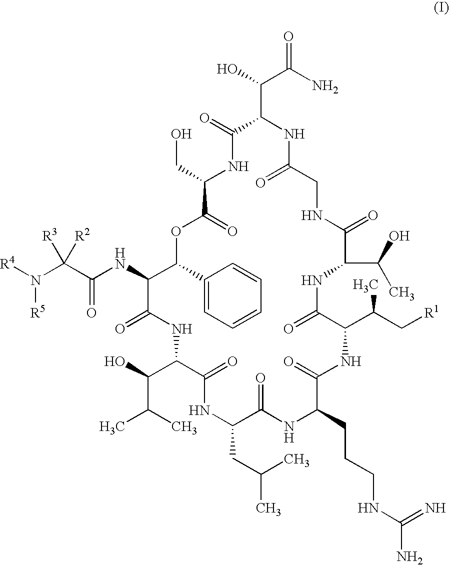
in which R¹ is H or CH₃,
in which R² is hydrogen, C₃-C₆-cycloalkyl, C₅-C₆-cycloalkenyl, C₃-C₆ cycloalkylmethyl, 5- to 7-membered heterocyclylmethyl, methyl, ethyl, n-propyl, isopropyl, 1-methylprop-1-yl, 2-methylprop-1-yl, 2,2-dimethylprop-1-yl, 1,1-dimethylprop-1-yl, 1-ethylprop-1-yl, 1-ethyl-1-methylprop-1-yl, n-butyl, 2-methylbut-1-yl, 3-methylbut-1-yl, 1-ethylbut-1-yl, tert-butyl, 4-methylpent-1-yl, n-hexyl, alkenyl or aryl,
whereby R² may be substituted with 0, 1, 2 or 3 substituents selected independently of one another from the group consisting of halogen, hydroxy, amino, cyano, trimethylsilyl, alkyl, alkoxy, benzyloxy, C₃-C₆-cycloalkyl, aryl, 5- to 10-membered heteroaryl, alkylamino, arylamino, alkylcarbonylamino, arylcarbonylamino, alkylcarbonyl, alkoxycarbonyl, arylcarbonyl and benzyloxycarbonylamino,
wherein aryl and heteroaryl in turn may be substituted with 0, 1, 2 or 3 substituents selected independently of one another from the group consisting of halogen, hydroxy, amino, cyano, nitro, alkyl, alkoxy and phenyl,
in which R³ is hydrogen or C₁-C₄-alkyl,
or
in which R² and R³ together with the carbon atom to which they are bonded form a C₃-C₆-cycloalkyl ring or a 5- to 7-membered heterocyclyl ring, whereby the cycloalkyl ring and the heterocyclyl ring may be substituted with 0, 1, 2 or 3 substituents selected independently of one another from the group consisting of trifluoromethyl, alkyl, alkoxy and alkylcarbonyl,
in which R⁴ is alkyl, C₃-C₆-cycloalkyl, 5- to 7-membered heterocyclyl, aryl, 5- or 6-membered heteroaryl, alkylcarbonyl, alkoxycarbonyl, C₃-C₆-cycloalkylcarbonyl, 5- to 7-membered heterocyclylcarbonyl, arylcarbonyl, 5- or 6-membered heteroarylcarbonyl or alkylaminocarbonyl,
whereby alkyl, cycloalkyl, heterocyclyl, aryl, heteroaryl, alkoxycarbonyl, cycloalkylcarbonyl, heterocyclylcarbonyl, arylcarbonyl, heteroarylcarbonyl and alkylaminocarbonyl may be substituted with 0, 1, 2 or 3 substituents selected independently of one another from the group consisting of halogen, hydroxy, amino, alkylamino and phenyl,
and
whereby alkylcarbonyl is substituted with an amino or alkylamino substituent,
and
whereby alkylcarbonyl may be substituted with a further 0, 1 or 2 substituents selected independently of one another from the group consisting of halogen, hydroxy, trimethylsilyl, alkoxy, alkylthio, benzyloxy, C₃-C₆-cycloalkyl, phenyl, naphthyl, 5- to 10-membered heteroaryl, alkylcarbonylamino, alkoxycarbonylamino, arylcarbonylamino, arylcarbonyloxy, benzyloxycarbonyl and benzyloxycarbonylamino,
whereby phenyl and heteroaryl in turn may be substituted with 0, 1, 2 or 3 substituents selected independently of one another from the group consisting of halogen, hydroxy, nitro, alkyl, alkoxy and phenyl,
or two substituents on the same carbon atom in the alkylcarbonyl together with the carbon atom to which they are bonded form a C₃-C₆-cycloalkyl ring or a 5- to 7-membered heterocyclyl ring,
whereby the cycloalkyl ring and the heterocyclyl ring may be substituted with 0, 1, 2 or 3 substituents selected independently of one another from the group consisting of trifluoromethyl, alkyl and alkoxy,
or
whereby the cycloalkyl ring may be benzo-fused,
in which R⁵ is hydrogen, C₁-C₄-alkyl, cyclopropyl or cyclopropylmethyl,
or
in which R⁴ and R⁵ together with the nitrogen atom to which they are bonded form a 5- to 7-membered heterocyclyl ring, whereby the heterocyclyl ring may be substituted with 0, 1, 2, or 3 substituents selected independently of one another from the group consisting of halogen, hydroxy, amino, cyano, alkyl, alkoxy and alkylamino,
by intramolecular cyclization of a compound of the following formula (II) 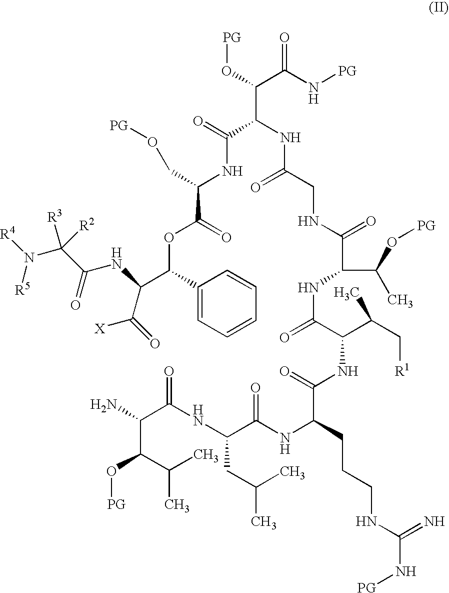
in which R¹ to R⁵ are as defined above,
in which X is OH, an active ester, a pseudohalogen or a halogen, and
in which PG is H or a suitable protecting group,
and subsequent deprotection of the cyclic intermediate to form said cyclic depsipeptide of formula (I).

2. The method of claim 1, whereby said compound of formula (II) is a compound of the following formula (IIa) 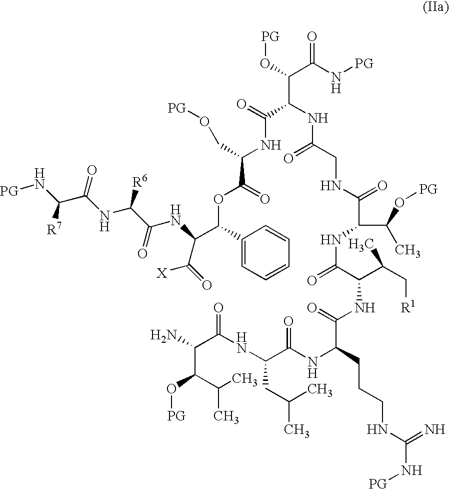
in which X and R¹ are as defined in claim 1,
in which R⁶ is isopropylmethyl, tert-butylmethyl, 2,2-dimethylbut-1-yl, 2-ethyl-2-methylbut-1-yl, 2,2-diethylbut-1-yl, 2,2-dimethylpent-1-yl, 3-pyridylmethyl, 4-trifluoromethyl-3-pyridylmethyl, benzyl or trimethylsilylmethyl,
in which R⁷ is isopropylmethyl, tert-butylmethyl, 2,2-dimethylbut-1-yl, 2-ethyl-2-methylbut-1-yl, 2,2-diethylbut-1-yl, 2,2-dimethylpent-1-yl, trimethylsilylmethyl or benzyl, and
in which PG is H or a suitable protecting group.

3. The method of claim 2, whereby
R⁶ is isopropylmethyl, tert-butylmethyl or 3-pyridylmethyl, and
R⁷ is isopropylmethyl, tert-butylmethyl or trimethylsilylmethyl.

4. The method of claim 3, whereby R⁶═R⁷ and is isopropylmethyl.

5. The method of claim 1, whereby X is OH.

6. The method of claim 1, whereby R¹ is CH₃.

7. The method of claim 1, further comprising the preparation of said compound of formula (II) by coupling a compound of the following formula (III) with a compound of the following formula (IV) 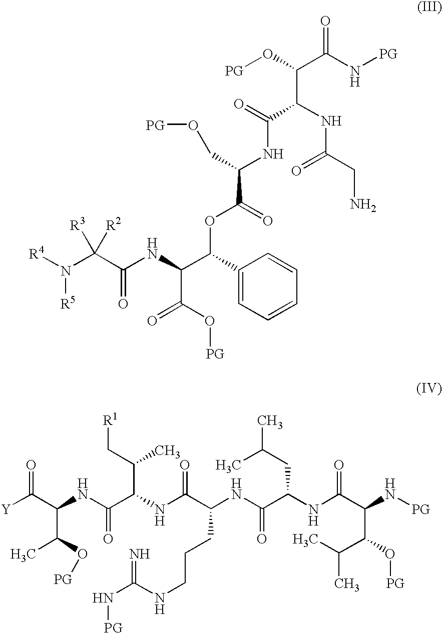
in which R¹ to R⁵ are as defined in claim 1,
in which Y is OH, an active ester, a pseudohalogen or a halogen, and
in which PG is H or a suitable protecting group,
and, where appropriate, partial or complete deprotection of the intermediate, as well as where appropriate conversion of the carboxy group of the 3-hydroxyphenylalanine into a group of formula —C(═O)X in which X is as defined in claim 1.

8. The method of claim 7, whereby said compound of formula (III) is a compound of the following formula (IIIa) 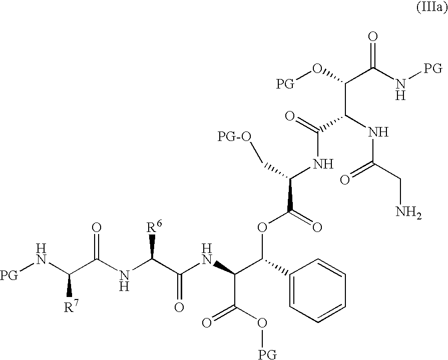
in which R⁶ and R⁷ are as defined in claim 2, and
in which PG is H or a suitable protecting group.

9. The method of claim 7, further comprising the preparation of said compound of formula (III) by coupling a compound of the following formula (V) with a compound of the following formula (VI) 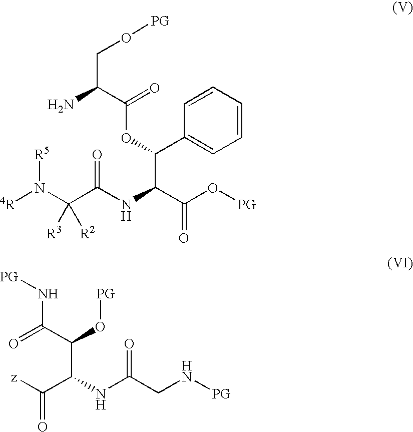
in which R² to R⁵ are as defined in claim 1,
in which Z is OH, an active ester, a pseudohalogen or a halogen, and
in which PG is H or a suitable protecting group,
and, where appropriate, partial or complete deprotection of the intermediate.

10. The method of claim 9, whereby said compound of formula (V) is a compound of the following formula (Va) 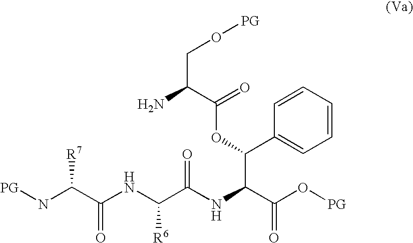
in which R⁶ and R⁷ are as defined in claim 2, and
in which PG is H or a suitable protecting group.

11. A compound of the following formula (III) 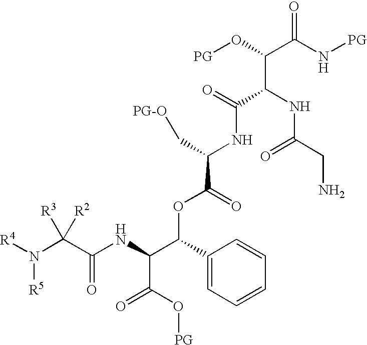
in which R² to R⁵ are as defined in claim 1, and
in which PG is H or a suitable protecting group.

12. The compound of claim 11, having the following formula (IIIa) 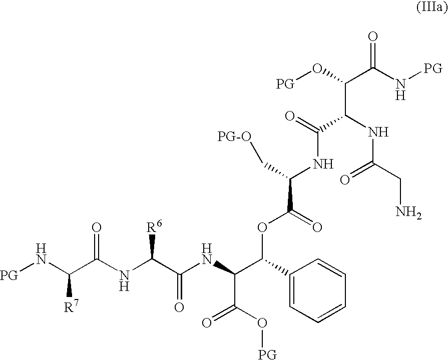
in which R⁶ and R⁷ are as defined in claim 2, and
in which PG is H or a suitable protective group.

13. The compound of claim 12, having the following formula (IIIb)

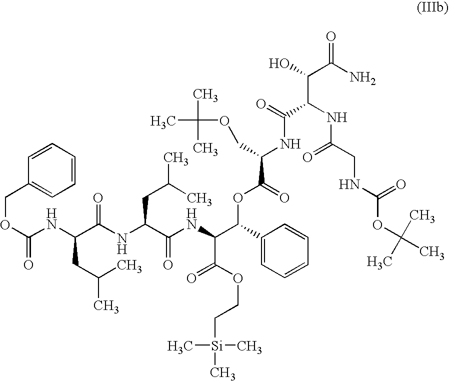

14. A method for preparing a compound of claim 11, comprising the coupling of a compound of formula (V) with a compound of formula (VI) 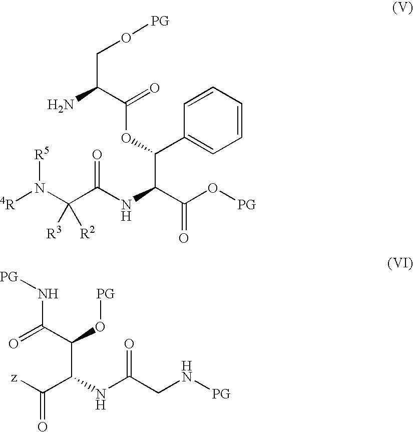
in which R² to R⁵ are as defined in claim 1,
in which Z is OH, an active ester, a pseudohalogen or a halogen, and
in which PG is H or a suitable protecting group,
and, where appropriate, partial or complete deprotection of the intermediate.

15. A compound of the following formula (VI) 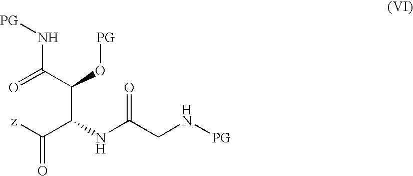
in which Z is OH, an active ester, a pseudohalogen or a halogen, and
in which PG is H or a suitable protecting group.

16. The compound of claim 15, having the following formula (VIa)

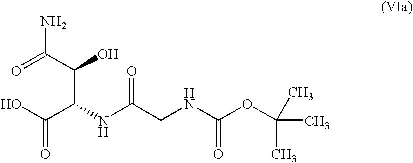

17. A method for preparing a compound of claim 15, comprising the coupling of a compound of the following formula (VII) with a compound of the following formula (VIII) 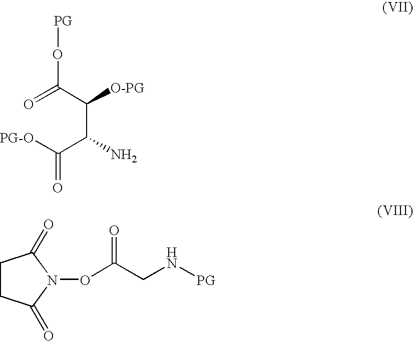
in which PG is H or a suitable protecting group,
and, where appropriate, complete or partial deprotection of the intermediate.

18. The method of claim 17, whereby said compound of formula (VII) is a compound of the following formula (VIIa).

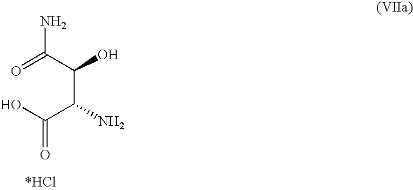

19. The method of claim 17, whereby said compound of formula (VIII) is a compound of the following formula (VIIIa). [chemical structure]
